(19) United States
(12) Patent Application Publication
(10) Pub. No.: US 2026/0174553 A1
(43) Pub. Date: Jun. 25, 2026

(54) LEAFLETS WITH POLYMERIC TISSUE-ADHERENT SURFACES

(71) Applicants: Tamir S. Levi, Zikhron Yaakov (IL); Michael Bukin, Pardes Hanna (IL)

(72) Inventors: Tamir S. Levi, Zikhron Yaakov (IL); Michael Bukin, Pardes Hanna (IL)

(73) Assignee: Edwards Lifesciences Corporation, Irvine, CA (US)

(21) Appl. No.: 19/541,940

(22) Filed: Feb. 17, 2026

Related U.S. Application Data
(63) Continuation of application No. PCT/US2024/042671, filed on Aug. 16, 2024.
(60) Provisional application No. 63/533,853, filed on Aug. 21, 2023.

(51) Int. Cl. A61F 2/24; A61F 2/00; A61L 27/18; A61L 27/36; A61L 27/56

(52) U.S. Cl. CPC A61F 2/2418; A61L 27/18; A61L 27/36; A61L 27/56; A61F 2002/0081; A61F 2002/0086; A61L 2400/04; A61L 2400/18; A61L 2430/20

(57) ABSTRACT

The present disclosure relates to prosthetic valves configured to allow or encourage tissue adherence to polymeric surfaces of valvular structures thereof. In an example, a prosthetic valve can include a frame movable between a radially compressed state and a radially expanded state, and a valvular structure mounted inside the frame, the valvular structure comprising a plurality of leaflets. Each leaflet comprises a leaflet polymeric inner surface facing a central longitudinal axis of the prosthetic valve, and a leaflet polymeric outer surface facing the frame, wherein at least one of the polymeric surfaces of the leaflet comprises a tissue-adherent surface, configured to allow tissue adherence thereto.

### CROSS-REFERENCE TO RELATED APPLICATIONS

This application is a continuation of International Application No. PCT/US2024/042671, filed Aug. 16, 2024, which claims the benefit of U.S. Patent Application No. 65/533,853, filed Aug. 21, 2023, the entire disclosures all of which are incorporated by reference for all purposes.

### FIELD

The present disclosure relates to prosthetic valves that include leaflets having polymeric surface regions configured to allow or encourage tissue adherence thereto.

### BACKGROUND

Native heart valves, such as the aortic, pulmonary and mitral valves, function to assure adequate directional flow from, and to, the heart, and between the heart's chambers, to supply blood to the whole cardiovascular system. Various valvular diseases can render the valves ineffective and require replacement with artificial valves. Surgical procedures can be performed to repair or replace a heart valve. Conventional surgically implantable prosthetic valve can include a leaflet assembly mounted within a relatively rigid support frame or ring. Components of the prosthetic valve are usually assembled with one or more biocompatible fabrics, and a fabric-covered sewing ring is provided around the valve for suturing to the tissue of the native leaflet.

Since surgeries are prone to an abundance of clinical complications, alternative less invasive techniques of delivering a prosthetic heart valve over a catheter and implanting it over the native malfunctioning valve have been developed over the years. Different types of prosthetic heart valves are known to date, including balloon expandable valve, self-expandable valves and mechanically-expandable valves.

Different methods of delivery and implantation are also known, and may vary according to the site of implantation and the type of prosthetic valve. One exemplary technique includes utilization of a delivery assembly for delivering a prosthetic valve in a crimped state, from an incision which can be located at the patient's femoral or iliac artery, toward the native malfunctioning valve. Once the prosthetic valve is properly positioned at the desired site of implantation, it can be expanded against the surrounding anatomy, such as an annulus of a native valve, and the delivery assembly can be retrieved thereafter.

### SUMMARY

Most expandable prosthetic valve include flexible leaflets attached to expandable frames thereof, wherein the leaflets are configured to transition between closed and open states, so as to regulate flow of blood through the prosthetic valves. In some cases, regions of the prosthetic valve might be subjected to blood stasis, low flow, or recirculation zones between the leaflets and the frame. Stagnant or otherwise disturbed pools of blood behind the leaflets can cause thrombus formations, loosely attached to a surface of the leaflet facing the frame. Some types of leaflets can be made of, or be coated by, polymeric materials, which can result in relatively smooth thromboresistant surfaces. In the case of smooth polymeric leaflet surfaces, which are thromboresistant or at least not formed to encourage tissue adherence thereto, such thrombotic deposits may occasionally detach from the leaflets and travel downstream, posing a risk of occluding narrower portions of the vasculature.

In one of its basic configurations, a prosthetic valve comprises a frame, movable between a radially compressed state and a radially expanded state, and a leaflet coupled to the frame, wherein the leaflet comprises a first edge, a second edge opposite to the first edge, and polymeric surfaces extending between the first and second edges. This basic configuration can preferably be provided with any one or more of the features described elsewhere herein, in particular with those of the examples described hereafter. However, it should be understood that the basic configuration can preferably also be provided with any one or more of the features shown in the figures and/or described in conjunction with the figures, either in addition to or alternatively to the features of the examples described hereafter.

In some examples, at least one of the leaflet polymeric surfaces comprises at least one tissue-adherent surface, configured to allow tissue adherence thereto.

In some examples, the first edge is a cusp edge and the second edge is a free edge.

In some examples, the polymeric surfaces comprise a leaflet polymeric inner surface which is oriented towards a central longitudinal axis of the prosthetic valve, and a leaflet polymeric outer surface which is oriented towards the frame.

In some examples, the leaflet is one of a plurality of leaflets of a valvular structure mounted inside the frame.

In some examples, the at least one tissue-adherent surface can optionally comprise thermoplastic polyurethane.

In some examples, the at least one tissue-adherent surface can optionally comprise a plurality of pores.

In some examples, the at least one tissue-adherent surface can optionally comprise at least one thrombogenic surface configured to allow adherence of tissue exceeding an average thickness of 200 μm thereto.

In some examples, the thrombogenic surface can optionally be configured to prevent spontaneous detachment of tissue adhered thereto for at least a predetermined period of time.

In some examples, at least 80% of the plurality of pores of the thrombogenic surface can optionally have an inscribed circle diameter of between 10 μm and 100 μm.

In some examples, the pores of the thrombogenic surface can optionally be formed between electrospun filaments.

In some examples, an electrospun layer defining the thrombogenic surface can optionally have a thickness between 50 μm and 200 μm.

In some examples, the leaflet polymeric outer surface can optionally comprise a distal outer surface portion extending between the cusp edge and a proximal edge of the distal outer surface portion, and a proximal outer surface portion extending between the proximal edge of the distal outer surface portion and the free edge.

In some examples, the distal outer surface portion can optionally define a textured outer portion height, which is less than a height defined by the proximal outer surface portion.

In some examples, the at least one tissue-adherent surface can optionally comprise at least one neointimal-formation encouraging surface configured to allow formation of neointimal tissue that does not exceed an average thickness of 200 μm.

In some examples, the leaflet polymeric outer surface can optionally comprise the at least one neointimal-formation encouraging surface.

In some examples, the entire surface area of the leaflet polymeric outer surface can optionally comprise the at least one neointimal-formation encouraging surface.

In some examples, the leaflet polymeric inner surface can optionally comprise the at least one neointimal-formation encouraging surface.

In some examples, the entire surface area of the leaflet polymeric inner surface can optionally comprise the at least one neointimal-formation encouraging surface.

In some examples, at least 80% of the plurality of pores of the at least one neointimal-formation encouraging surface can optionally have an inscribed circle diameter of between 3 μm and 50 μm.

In some examples, the pores of the at least one neointimal-formation encouraging surface can optionally be formed between electrospun filaments.

In some examples, an electrospun layer defining the at least one neointimal-formation encouraging surface can optionally have a thickness between 10 μm and 50 μm.

In some examples, each leaflet can optionally comprise a base layer between the leaflet polymeric inner surface and the leaflet polymeric outer surface.

In some examples, the base layer can optionally comprise a polymeric material which is different from the polymeric material comprised in the leaflet polymeric surfaces.

In some examples, the base layer can optionally comprise a fabric.

In some examples, the base layer can optionally comprise a tissue.

The aspects of this disclosure can be used in combination or separately. This summary is provided to introduce a selection of concepts in a simplified form that are further described below in the detailed description. This summary is not intended to identify key features or essential features of the claimed subject matter, nor is it intended to be used to limit the scope of the claimed subject matter. The foregoing and other objects, features, and advantages will become more apparent from the following detailed description, which proceeds with reference to the accompanying figures.

### BRIEF DESCRIPTION OF THE FIGURES

Some examples are described herein with reference to the accompanying figures. The description, together with the figures, makes apparent to a person having ordinary skill in the art how some examples may be practiced. The figures are for the purpose of illustrative description and no attempt is made to show structural details of an example in more detail than is necessary for a fundamental understanding of the disclosure. For the sake of clarity, some objects depicted in the figures are not to scale.

In the Figures:

FIG. 1A is a perspective view of an exemplary prosthetic valve.

FIG. 1B is a perspective view of the prosthetic valve of FIG. 1A, without the outer skirt.

FIG. 1C is a perspective view of a frame of the prosthetic valve of FIGS. 1A-1B.

FIG. 2 is a perspective view of an example of a valvular structure that includes three leaflets having thromboresistant polymeric surfaces.

FIG. 3 shows an exemplary delivery apparatus carrying an exemplary balloon expandable prosthetic valve.

FIG. 4 is a cross-sectional view of an exemplary prosthetic valve, showing thrombotic dislodgements from leaflets thereof.

FIGS. 5A and 5B are views in perspective of an exemplary leaflet having a leaflet outer surface that includes a distal outer surface portion.

FIG. 6A is a cross-sectional view of an exemplary prosthetic valve that includes the leaflets of FIGS. 5A-5B.

FIG. 6B is a cross-sectional view of the prosthetic valve of FIG. 6A, with thromboses attached to the distal outer surface portions of the leaflets.

FIG. 7 is a view in perspective of a portion of an exemplary leaflet that includes both a distal outer surface portion and a distal inner surface portion.

FIG. 8A is a cross-sectional view of an exemplary prosthetic valve that includes the leaflets of FIG. 7.

FIG. 8B shows a cross-sectional view of the prosthetic valve of FIG. 8A, with thromboses attached to the distal outer surface portions and the distal inner surface portions of the leaflets.

FIG. 9 is a perspective view of an exemplary leaflet comprising a base layer sandwiched between two polymeric layers.

FIG. 10 shows a flattened view of an exemplary leaflet that includes a distal outer surface portion and a distal inner surface portion having non-uniform heights.

FIG. 11 is a cross-sectional view of an exemplary prosthetic valve that includes a strip attached to an inner skirt of the valve.

FIG. 12 is a cross-sectional view of an exemplary prosthetic valve that includes a strip attached to the frame of the valve.

FIG. 13 is a cross-sectional view of an exemplary prosthetic valve that includes a valvular structure directly coupled to the frame of the valve.

FIG. 14A is a view in perspective of an exemplary leaflet having both thrombotic surfaces and neointimal-formation encouraging surfaces, shown from an outer view angle.

FIG. 14B is an enlarged view of a distal outer surface portion of FIG. 14A.

FIG. 14C is an enlarged view of a proximal outer surface portion of FIG. 14A.

FIG. 14D is a view in perspective of the leaflet of FIG. 14A, shown from a side/inner view angle.

FIG. 15 is a cross-sectional view of an exemplary prosthetic valve that includes the leaflets of FIGS. 14A-14D, showing thromboses attached to the thrombotic surfaces and tissue layers formed over the neointimal-formation encouraging surfaces.

FIGS. 16A and 16B are views in perspective of an exemplary leaflet having neointimal-formation encouraging surfaces, without including thrombotic surfaces.

FIG. 17 is a cross-sectional view of an exemplary prosthetic valve that includes the leaflets of FIGS. 16A-16B, showing tissue layers formed over the leaflet surfaces.

### DETAILED DESCRIPTION

For purposes of this description, certain aspects, advantages, and novel features of the examples of this disclosure are described herein. The disclosed methods, apparatus, and systems should not be construed as being limiting in any way. Instead, the present disclosure is directed toward all novel and nonobvious features and aspects of the various disclosed examples, alone and in various combinations and sub-combinations with one another. The methods, apparatus, and systems are not limited to any specific aspect or feature or combination thereof, nor do the disclosed examples require that any one or more specific advantages be present, or problems be solved. The technologies from any example can be combined with the technologies described in any one or more of the other examples. In view of the many possible examples to which the principles of the disclosed technology may be applied, it should be recognized that the illustrated examples are only preferred examples and should not be taken as limiting the scope of the disclosed technology.

Although the operations of some of the disclosed examples are described in a particular, sequential order for convenient presentation, it should be understood that this manner of description encompasses rearrangement, unless a particular ordering is required by specific language set forth below. For example, operations described sequentially may in some cases be rearranged or performed concurrently. Moreover, for the sake of simplicity, the attached figures may not show the various ways in which the disclosed methods can be used in conjunction with other methods. Additionally, the description sometimes uses terms like “provide” or “achieve” to describe the disclosed methods. These terms are high-level abstractions of the actual operations that are performed. The actual operations that correspond to these terms may vary depending on the particular implementation and are readily discernible by one of ordinary skill in the art.

All features described herein are independent of one another and, except where structurally impossible, can be used in combination with any other feature described herein.

As used in this application and in the claims, the singular forms “a,” “an,” and “the” include the plural forms unless the context clearly dictates otherwise. Additionally, the terms “have” or “includes” means “comprises”. Further, the terms “coupled”, “connected”, and “attached”, as used herein, are interchangeable and generally mean physically, mechanically, chemically, magnetically, and/or electrically coupled or linked and does not exclude the presence of intermediate elements between the coupled or associated items absent specific contrary language. As used herein, “and/or” means “and” or “or”, as well as “and” and “or”.

Directions and other relative references may be used to facilitate discussion of the drawings and principles herein, but are not intended to be limiting. For example, certain terms may be used such as “inner,” “outer,” “upper,” “lower,” “inside,” “outside,”, “top,” “bottom,” “interior,” “exterior,” “left,” right,” and the like. Such terms are used, where applicable, to provide some clarity of description when dealing with relative relationships, particularly with respect to the illustrated examples. Such terms are not, however, intended to imply absolute relationships, positions, and/or orientations. For example, with respect to an object, an “upper” part can become a “lower” part simply by turning the object over. Nevertheless, it is still the same part and the object remains the same.

The term “plurality” or “plural” when used together with an element means two or more of the element. Directions and other relative references (e.g., inner and outer, upper and lower, above and below, left and right, and proximal and distal) may be used to facilitate discussion of the drawings and principles herein but are not intended to be limiting.

The terms “proximal” and “distal” are defined relative to the use position of a delivery apparatus. In general, the end of the delivery apparatus closest to the user of the apparatus is the proximal end, and the end of the delivery apparatus farthest from the user (e.g., the end that is inserted into a patient's body) is the distal end. The term “proximal” when used with two spatially separated positions or parts of an object can be understood to mean closer to or oriented towards the proximal end of the delivery apparatus. The term “distal” when used with two spatially separated positions or parts of an object can be understood to mean closer to or oriented towards the distal end of the delivery apparatus. The terms “longitudinal” and “axial” are interchangeable, and refer to an axis extending in the proximal and distal directions, unless otherwise expressly defined.

It should be understood that the disclosed examples can be adapted to deliver inflatable balloons, and in some implementations, to deliver and implant prosthetic devices expandable by such inflatable balloons, to and/or in any of the native annuluses of the heart (e.g., the aortic, pulmonary, mitral, and tricuspid annuluses), and can be used with any of various delivery approaches (e.g., retrograde, antegrade, transseptal, transventricular, transatrial, transapical etc.).

Throughout the figures of the drawings, different superscripts for the same reference numerals are used to denote different examples of the same elements. Examples of the disclosed devices and systems may include any combination of different examples of the same elements. Specifically, any reference to an element without a superscript may refer to any alternative example of the same element denoted with a superscript. In order to avoid undue clutter from having too many reference numbers and lead lines on a particular drawing, some components will be introduced via one or more drawings and not explicitly identified in every subsequent drawing that contains that component.

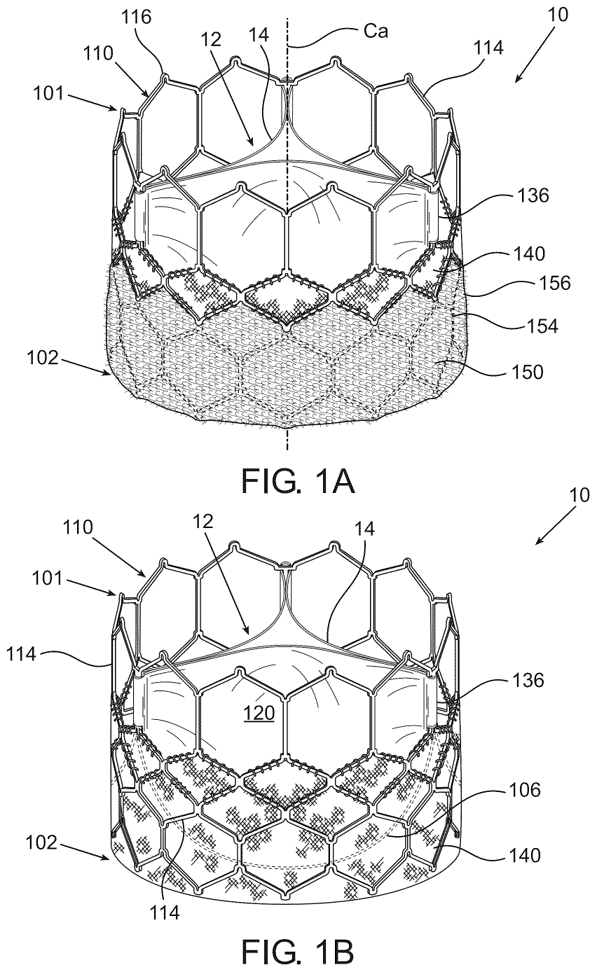

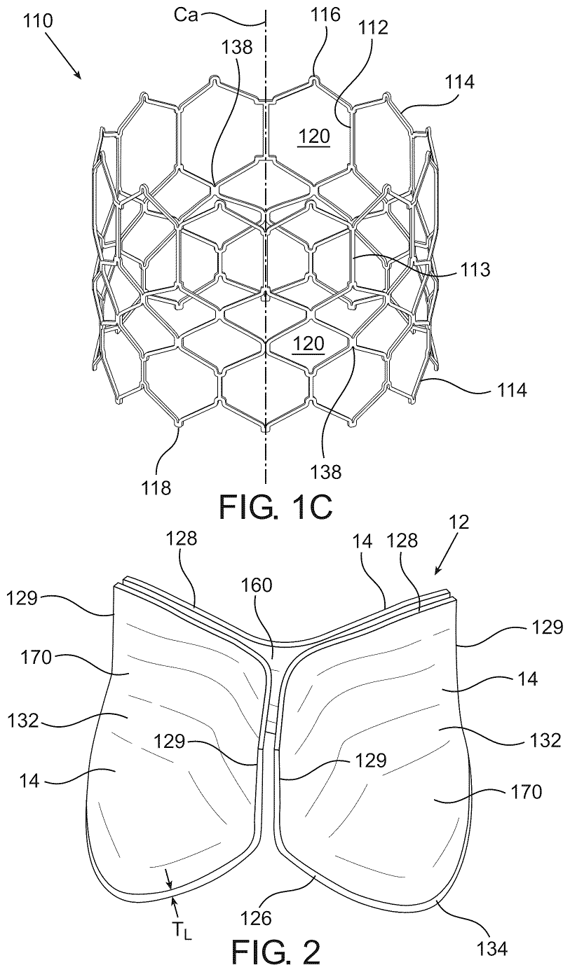

FIGS. 1A and 1B show perspective views of an exemplary prosthetic valve 10, with and without an outer skirt 150 surrounding the frame 110, respectively. FIG. 1C shows the frame 110 without any other soft components attached thereto. The term “prosthetic valve”, as used herein, refers to any type of a prosthetic valve deliverable to a patient's target site over a catheter, which is radially expandable and compressible between a radially compressed, or crimped, state, and a radially expanded state. Thus, the prosthetic valve can be crimped on or retained by an implant delivery apparatus (not shown) in the radially compressed state during delivery, and then expanded to the radially expanded state once the prosthetic valve reaches the implantation site. The expanded state may include a range of diameters to which the valve may expand, between the compressed state and a maximal diameter reached at a fully expanded state. Thus, a plurality of partially expanded states may relate to any expansion diameter between radially compressed or crimped state, and maximally expanded state. A prosthetic valve of the current disclosure (e.g., prosthetic valve 10, 100) may include any prosthetic valve configured to be mounted within the native aortic valve, the native mitral valve, the native pulmonary valve, and the native tricuspid valve.

It is understood that the prosthetic valves disclosed herein may be used with a variety of implant delivery apparatuses. Balloon expandable valves generally involve a procedure of inflating a balloon within a prosthetic valve, thereby expanding the prosthetic valve within the desired implantation site. Once the valve is sufficiently expanded, the balloon is deflated and retrieved along with a delivery apparatus (not shown). Self-expandable valves include a frame that is shape-set to automatically expand as soon an outer retaining shaft or capsule (not shown) is withdrawn proximally relative to the prosthetic valve. Mechanically expandable valves are a category of prosthetic valves that rely on a mechanical actuation mechanism for expansion. The mechanical actuation mechanism usually includes a plurality of expansion and locking assemblies (such as the prosthetic valves described in U.S. Pat. No. 10,603,165, International Application No. PCT/US2021/052745, US. Patent Application Publication No. 2023/0225863A1, and U.S. Patent Application Publication No. 2024/0207046A1, the entire disclosures all of which are incorporated by reference), releasably coupled to respective actuation assemblies of a delivery apparatus, controlled via a handle (not shown) for actuating the expansion and locking assemblies to expand the prosthetic valve to a desired diameter. The expansion and locking assemblies may optionally lock the valve's diameter to prevent undesired recompression thereof, and disconnection of the actuation assemblies from the expansion and locking assemblies, to enable retrieval of the delivery apparatus once the prosthetic valve is properly positioned at the desired site of implantation.

FIGS. 1A-1C show an example of a prosthetic valve 10, which can be a balloon expandable valve or any other type of valve, illustrated in an expanded state. The prosthetic valve 10 can comprise an outflow end 101 and an inflow end 102. In some instances, the outflow end 101 is the proximal end of the prosthetic valve 10, and the inflow end 102 is the distal end of the prosthetic valve 10. Alternatively, depending for example on the delivery approach of the valve, the outflow end can be the distal end of the prosthetic valve, and the inflow end can be the distal end of the proximal valve.

The term “outflow”, as used herein, refers to a region of the prosthetic valve through which the blood flows through and out of the prosthetic valve 10.

The term “inflow”, as used herein, refers to a region of the prosthetic valve through which the blood flows into the prosthetic valve 10.

In the context of the present application, the terms “lower” and “upper” are used interchangeably with the terms “inflow” and “outflow”, respectively. Thus, for example, the lower end of the prosthetic valve is its inflow end and the upper end of the prosthetic valve is its outflow end.

In the context of the present application, the terms “lower” and “upper” are used interchangeably with the terms “distal to” and “proximal to”, respectively. Thus, for example, a lowermost component can refer to a distal-most component, and an uppermost component can similarly refer to a proximal-most component.

The terms “longitudinal” and “axial”, as used herein, refer to an axis extending in the proximal and distal directions, unless otherwise expressly defined.

The prosthetic valve 10 comprises an annular frame 110 movable between a radially compressed configuration and a radially expanded configuration, and a valvular structure 12 mounted within the frame 110. The frame 110 can be made of various suitable materials, including plastically-deformable materials such as, but not limited to, stainless steel, a nickel-based alloy (e.g., a nickel-cobalt-chromium alloy such as MP35N™ alloy (SPS Technologies), polymers, or combinations thereof. When constructed of a plastically-deformable materials, the frame 110 can be crimped to a radially compressed state on a balloon catheter, and then expanded inside a patient by an inflatable balloon or equivalent expansion mechanism. Alternatively or additionally, the frame 110 can be made of shape-memory materials such as, but not limited to, nickel-titanium alloy (e.g., nitinol). When constructed of a shape-memory material, the frame 110 can be crimped to a radially compressed state and restrained in the compressed state by insertion into a shaft or equivalent mechanism of a delivery apparatus.

In the example illustrated in FIGS. 1A-1C, the frame 110 is an annular, stent-like structure comprising a plurality of intersecting struts 114. In this application, the term “strut” encompasses axial struts, angled struts, laterally extendable struts, commissure windows, commissure support struts, support posts, and any similar structures described by U.S. Pat. Nos. 7,993,394 and 9,393,110, which are incorporated herein by reference. A strut 114 may be any elongated member or portion of the frame 110. The frame 110 can include a plurality of strut rungs that can collectively define one or more rows of cells 120. The frame 110 can have a cylindrical or substantially cylindrical shape having a constant diameter from the inflow end 102 to the outflow end 101 as shown, or the frame can vary in diameter along the height of the frame, as disclosed in U.S. Pat. No. 9,155,619, which is incorporated herein by reference.

The end portions of the struts 114 are forming apices 116 at the outflow end 101 and apices 118 at the inflow end 102. The struts 114 can intersect at additional junctions 138 formed between the outflow apices 116 and the inflow apices 118. The junctions 138 can be equally or unequally spaced apart from each other, and/or from the apices 116, 118, between the outflow end 101 and the inflow end 102.

The struts 114 can include a plurality of angled struts and vertical or axial struts. FIGS. 1A-1C show an exemplary prosthetic valve 10 that can be representative of, but is not limited to, a balloon expandable prosthetic valve. The frame 110 of the prosthetic valve 10 illustrated in FIG. 1C comprises rungs of angled struts and axial struts disposed between some of the rungs of the angled struts. In such implementations of the frame, the struts can be pivotable or bendable relative to each other, so as to permit frame expansion or compression. For example, the frame 110 can include a single piece of material, such as a metal tube, via various processes such as, but not limited to, laser cutting, electroforming, and/or physical vapor deposition, while retaining the ability to collapse/expand radially in the absence of hinges and like.

A valvular structure 12, shown also for example in FIG. 2, can include a plurality of leaflets 14 (e.g., three leaflets), positioned at least partially within the frame 110, and configured to regulate flow of blood through the prosthetic valve 10 from the inflow end 102 to the outflow end 101. While three leaflets 14 arranged to collapse in a tricuspid arrangement, are shown in the example illustrated in FIGS. 1A-1B and 2, it will be clear that a prosthetic valve 10 can include any other number of leaflets 14. Adjacent leaflets 14 can be arranged together to form commissures 136 that are coupled (directly or indirectly) to respective portions of the frame 110, thereby securing at least a portion of the valvular structure 12 to the frame 110. Further details regarding transcatheter prosthetic valves, including the manner in which the valvular structures 12 can be coupled to the frame 110 of the prosthetic valve 10, can be found, for example, in U.S. Pat. Nos. 6,730,118, 7,393,360, 7,510,575, 7,993,394, 8,652,202, and 11,135,056, all of which are incorporated herein by reference in their entireties.

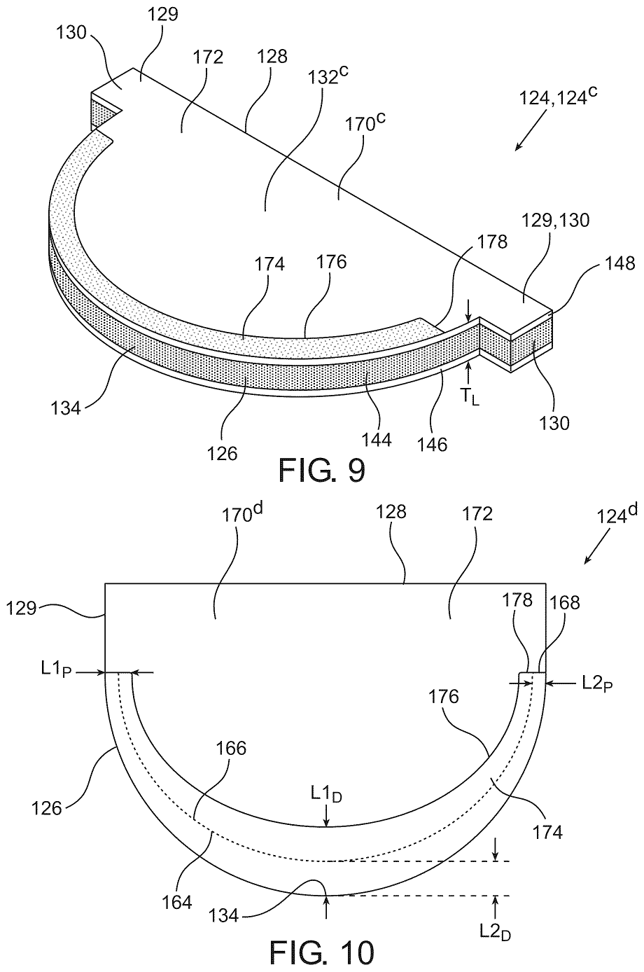

As shown for example in FIG. 2, three separate leaflets 14 can collectively define the valvular structure 12 in some examples. Each leaflet 14 can have a rounded cusp edge 126 opposite a free edge 128, and a pair of generally oppositely-directed commissure attachment portions 129 separating the cusp edge 126 and the free edge 128. The cusp edge 126 in such cases forms a single scallop. While commissure attachment portions 129 are illustrated in FIG. 2 as relatively linear portions of the leaflet 14 extending between the cusp edge 126 and the free edge 128, it is to be understood that exemplary leaflet 14 disclosed herein can include, in some examples, commissure attachment portions 129 in the form of tabs 130 extending in opposite directions, as illustrated for example in FIG. 9. Each leaflet 14 further comprises a leaflet inner surface 160, defined as the surface facing the valve central axis Ca (indicated in FIG. 1A), and a leaflet outer surface 170, opposite thereto so as to face the frame 110 when mounted inside the frame 110. The leaflet 14 has a leaflet thickness T_{L} defined between the leaflet inner surface 160 and the leaflet outer surface 170.

When such leaflets 14 are coupled to the frame and to each other, the lower edge of the resulting valvular structure 12 desirably has an undulating, curved scalloped shape. By forming the leaflets with this scalloped geometry, stresses on the leaflets 14 are reduced which, in turn, improves durability of the prosthetic valve. Moreover, by virtue of the scalloped shape, folds and ripples at the belly of each leaflet, which can cause early calcification in those areas, can be eliminated or at least minimized. The scalloped geometry also reduces the amount of tissue material used to form the valvular structure, thereby allowing a smaller, more even crimped profile at the inflow end of the valve.

The leaflets 14 can define a non-planar coaptation plane (not annotated) when their free edges 128 coapt with each other to seal blood flow through the prosthetic valve 10. Leaflets 14 can be secured to one another at their commissure attachment portions 129 to form commissures 136 of the valvular structure 12, which can be secured, directly or indirectly, to structural elements connected to the frame 110 or integrally formed as portions thereof, such as commissure posts or struts, commissure windows, and the like. When secured to two other leaflets 14 to form valvular structure 12, the cusp edges 126 of the leaflets 14 collectively form the scalloped line 106 of the valvular structure 12. Each leaflet 14 comprises a leaflet body 132, defined between the line of attachment of the leaflet to the frame, for example along scalloped line 106, and the free edge 128. The leaflet body 132 defines the movable portion of the leaflet 14, free to move toward the frame 110 in an open state of the valvular structure 12, and toward central axis Ca to coapt with other leaflets 14 in a closed state of the valvular structure 12. The lowest or distal-most end of each leaflet 14 is at cusp edge midpoint 134, which in turn also defines the lowest or distal-most region of scalloped line 106.

Any leaflet disclosed herein comprises at least one polymeric surface, and optionally two polymeric surfaces on opposite sides thereof. In some examples, the leaflet inner surface 160 is a leaflet inner polymeric surface. In some examples, the leaflet outer surface 170 is a leaflet outer polymeric surface. In some examples, leaflet 14 can be made of a polymeric material which defines both the inner 160 and outer 170 polymeric surfaces of the leaflet 14. In some examples, leaflet 14 can be coated by a polymeric material. For example, a leaflet 14 can be a multi-layered or coated leaflet that includes of a core material, which can comprise polymeric material, fabric, or tissue, wherein the core material can be coated by a polymeric layer over its inner surface so as to define the leaflet's polymeric inner surface 160, and/or over its outer surface so as to define the leaflet's polymeric outer surface 170.

Polymeric surfaces of leaflets 14 of valvular structure 12 can be, in some examples, relatively smooth or otherwise configured to be relatively thromboresistant. For example, a polymeric surface, which can be either a leaflet inner surface 160 and/or a leaflet outer surface 170, can include at least one or more of a thermoplastic polyurethane (TPU), a polytetrafluoroethylene (PTFE), an ultra-high molecular weight polyethylene (UHMWPE). This can be the result of a leaflet made of a polymeric material, or a leaflet comprising a core material coated by TPU, UHMPE, PTFE, or other suitable polymeric materials. The polytetrafluoroethylene (PTFE) may comprise expanded polytetrafluoroethylene (ePTFE) in some examples as desired. Various other thromboresistant materials may be utilized as desired. The leaflet polymeric inner 160 and/or outer 170 surface can include a smooth texture to be thromboresistant and inhibit tissue growth. In some examples, the leaflet 14 is made of a TPU film having a leaflet thickness T_{L} in a range between about 10 micrometers (μm) and about 300 μm.

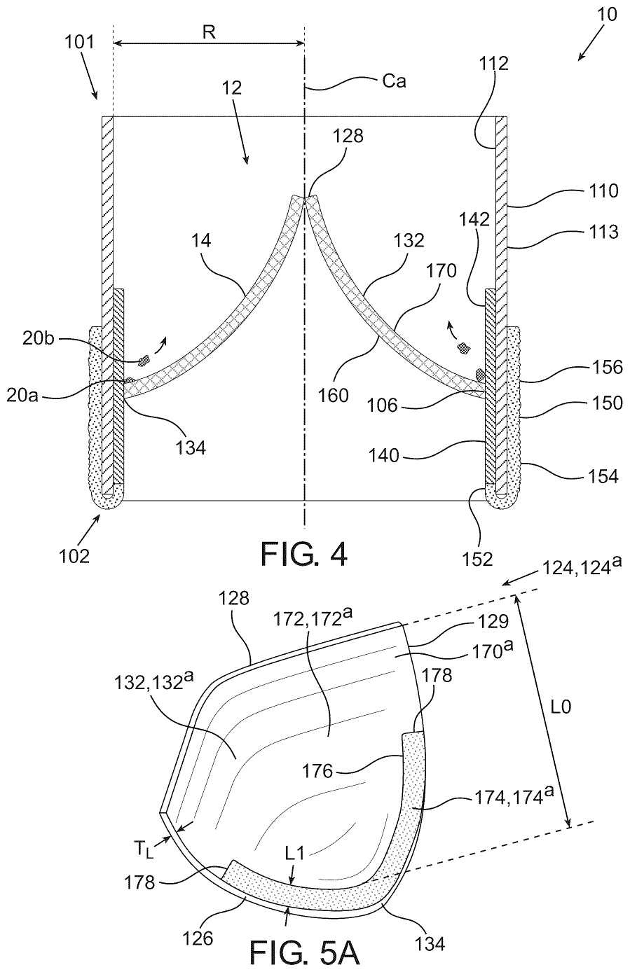

In some examples, the prosthetic valve 10 can comprise at least one skirt or sealing member. FIGS. 1A-1B show an example of a prosthetic valve 10 that includes an inner skirt 140, which can be secured to the inner surface 112 (annotated, for example, in FIG. 4) of the frame 110. Such an inner skirt 140 can be configured to function, for example, as a sealing member to prevent or decrease perivalvular leakage. An inner skirt 140 can further function as an anchoring region for valvular structure 12 to the frame 110, and/or function to protect the leaflets 14 against damage which may be caused by contact with the frame 110, for example during valve crimping or during working cycles of the prosthetic valve 10. FIG. 1B shows an inner skirt 140 disposed around and attached to the inner surface 112 of frame 110, wherein the valvular structure 12 is sutured to the inner skirt 140 along scalloped line 106. The inner skirt 140 can be coupled to the frame 110 via sutures or another form of coupler.

The prosthetic valve 10 can comprise, in some examples, an outer skirt 150 mounted on the outer surface 113 (annotated, for example, in FIG. 4) of frame 110, configure to function, for example, as a sealing member retained between the frame 110 and the surrounding tissue of the native annulus against which the prosthetic valve is mounted, thereby reducing risk of paravalvular leakage (PVL) past the prosthetic valve 10. The outer skirt 150 can be coupled to the frame 110 via sutures or another form of coupler.

Any of the inner skirt 140 and/or outer skirt 150 can be made of various suitable biocompatible materials, such as, but not limited to, various synthetic materials (e.g., PET) or natural tissue (e.g. pericardial tissue). In some cases, the inner skirt 140 can be formed of a single sheet of material that extends continuously around the inner surface 112 of frame 110. In some cases, the outer skirt 150 can be formed of a single sheet of material that extends continuously around the outer surface 113 of frame 110.

The outer skirt 150 can define an internal surface (not annotated) facing and optionally contacting the outer surface 113 of the frame 110, and an opposite external surface 156 facing away from the frame 110, toward the surrounding anatomy when implanted in a patient's body. In some examples, as illustrated for example in a cross-sectional view of prosthetic valve 10 in FIG. 4, the outer skirt can include an outer portion 154 extending over at least a portion of the outer surface 113 of frame 110, and fold over the frame 110 along the inflow end 102 to further include an inner portion 152 extending over a portion of the inner surface 112 of frame 110, extending over a limited height between the inflow end 102 and the inner skirt 140, for example. In such cases, the external surface 156 of the outer skirt 150 can be defined only over the outer portion 154, and not the inner portion 152. Such configurations, in which the outer skirt 150 covers inflow apices 118, can advantageously provide an atraumatic inflow end 102 of the prosthetic valve 10, preventing the inflow apices 118 from accidentally engaging or snagging portions of the delivery apparatus 200 during advancement through the patient's vasculature to the site of implantation.

While an outer skirt 150 is illustrated throughout some of the drawings of the current specification, such as FIGS. 4-6, to fold over inflow apices 118 so as to include both an inner portion 152 and an outer portion 154, it is to be understood that this configuration is shown by way of illustration and not limitation, and that an outer skirt 150 can similarly extend only over the outer surface 113 of the frame 110, such that it includes the outer portion 154 and does not necessarily define an inner portion 152.

In some examples, the outer skirt 150 is configured to allow tissue ingrowth, at least along outer portion 154. In some examples, the outer skirt 150 can include polyethylene terephthalate (PET) or another form of material that is configured to allow tissue ingrowth. In some examples, a coating may be utilized with the outer skirt 150 that is configured to allow tissue ingrowth. For example, a porous coating or other form of coating may be applied to a thromboresistant material such as ultra-high molecular weight polyethylene (UHMWPE) to allow for tissue ingrowth. The underlying material of the outer skirt 150 in such examples, may be a material that inhibits tissue ingrowth, yet is coated with a coating that allows tissue ingrowth, at least over outer portion 154.

In some examples, the material of the outer skirt 150 may be fabricated to allow tissue ingrowth. For example, a knit pattern of the material of the outer skirt 150 may be configured to allow tissue ingrowth, with a large knit pattern or other forms of knit patterns. A knit PET fabric may be utilized in some examples. In some examples, the outer skirt 150 may include yarns (e.g., textured yarns) extending radially outward. The material of the outer skirt 150 may include a porous texture to allow for tissue ingrowth. Combinations of such features may be utilized to result in an outer skirt 150 that allows for tissue ingrowth. It is to be understood that for implementations of outer skirts 150 that include both an inner portion 152 and an outer portion 154, any of the examples described hereinabove with respect to encouraging tissue ingrowth may be applied to the outer portion 154, while the inner portion 152 may or may not be configured to encourage tissue ingrowth.

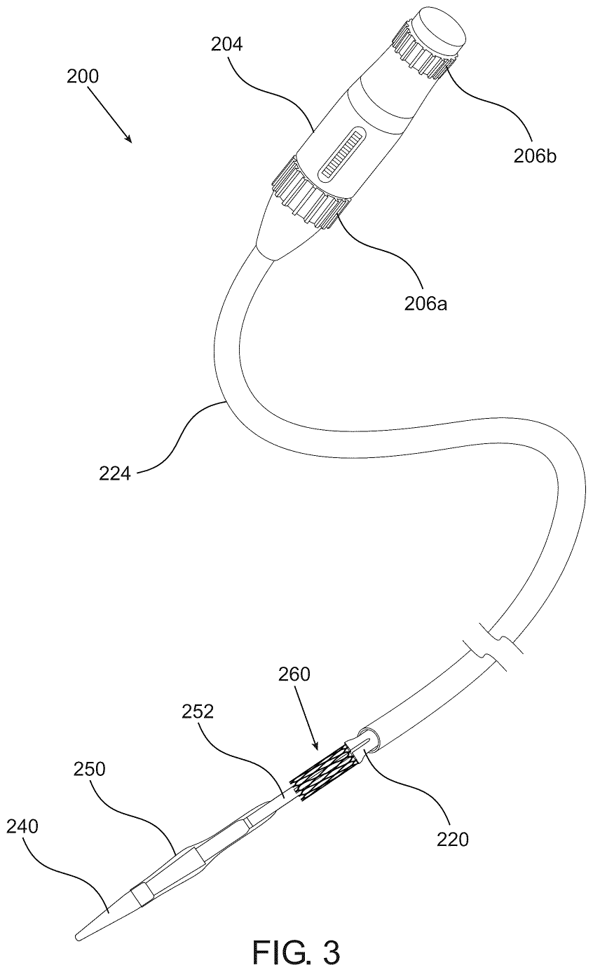

FIG. 3 illustrates a delivery apparatus 200, according to an exemplary configuration, adapted to deliver a balloon expandable prosthetic valve 260 described herein (e.g., prosthetic valve 10 or 100). It should be understood that the delivery apparatus 200 can be used to implant prosthetic devices other than prosthetic valves, such as stents or grafts.

The delivery apparatus 200 includes a handle 204 and a balloon catheter 252 having an inflatable balloon 250 mounted on its distal end. The prosthetic valve 260 can be carried in a crimped state over the balloon catheter 252. Optionally, an outer delivery shaft 224 can concentrically extend over the balloon catheter 252, and a push shaft 220 can be disposed over the balloon catheter 252, optionally between the balloon catheter 252 and the outer delivery shaft 224.

The outer delivery shaft 224, the push shaft 220, and the balloon catheter 252, can be configured to be axially movable relative to each other. For example, a proximally oriented movement of the outer delivery shaft 224 relative to the balloon catheter 252, or a distally oriented movement of the balloon catheter 252 relative to the outer delivery shaft 224, can expose the prosthetic valve 260 from the outer delivery shaft 224. The delivery apparatus 200 can further include a nosecone 240 carried by a nosecone shaft (hidden from view in FIG. 3) extending through a lumen of the balloon catheter 252.

The proximal ends of the balloon catheter 252, the outer delivery shaft 224, the push shaft 220, and optionally the nosecone shaft, can be coupled to the handle 204. During delivery of the prosthetic valve 260, the handle 204 can be maneuvered by an operator (e.g., a clinician or a surgeon) to axially advance or retract components of the delivery apparatus 200, such as the nosecone shaft, the balloon catheter 252, the outer delivery shaft 224, and/or the push shaft 220, through the patient's vasculature, as well as to inflate the balloon 250 mounted on the balloon catheter 252, so as to expand the prosthetic valve 260, and to deflate the balloon 250 and retract the delivery apparatus 200 once the prosthetic valve 260 is mounted in the implantation site.

The handle 204 can include a steering mechanism configured to adjust the curvature of the distal end portion of the delivery apparatus 200. In the illustrated example, the handle 204 includes an adjustment member, such as the illustrated rotatable knob 206^{a}, which in turn is operatively coupled to the proximal end portion of a pull wire. The pull wire can extend distally from the handle 204 through the outer delivery shaft 224 and has a distal end portion affixed to the outer delivery shaft 224 at or near the distal end of the outer delivery shaft 224. Rotating the knob 206^{a} can increase or decrease the tension in the pull wire, thereby adjusting the curvature of the distal end portion of the delivery apparatus 200. Further details on steering or flex mechanisms for the delivery apparatus can be found in U.S. Pat. No. 9,339,384, which is incorporated by reference herein. The handle 204 can further include an adjustment mechanism including an adjustment member, such as the illustrated rotatable knob 206^{b}. The adjustment mechanism can be configured to adjust the axial position of the push shaft 220 relative to the balloon catheter.

The prosthetic valve 260 can be carried by the delivery apparatus 200 during delivery in a crimped state, and expanded by balloon inflation to secure it in a native heart valve annulus. In an exemplary implantation procedure, the prosthetic valve 260 is initially crimped over the balloon catheter 252, proximal to the inflatable balloon 250. Because prosthetic valve 260 is crimped at a location different from the location of balloon 250, prosthetic valve 260 can be crimped to a lower profile than would be possible if it was crimped on top of balloon 250. This lower profile permits the clinician to more easily navigate the delivery apparatus 200 (including crimped prosthetic valve 260) through a patient's vasculature to the treatment location. The lower profile of the crimped prosthetic valve is particularly helpful when navigating through portions of the patient's vasculature which are particularly narrow, such as the iliac artery.

The balloon 250 can be secured to balloon catheter 252 at its balloon proximal end, and to either the balloon catheter 252 or the nosecone 240 at its distal end. The distal end portion of the push shaft 220 is positioned proximal to the outflow end (e.g., outflow end 101) of the prosthetic valve 260.

When reaching the site of implantation, and prior to balloon inflation, the push shaft 220 is advanced distally, allowing its distal end portion to contact and push against the outflow end of prosthetic valve 260, pushing the valve 260 distally therewith. The distal end of push shaft 220 is dimensioned to engage with the outflow end of the prosthetic valve 260 in a crimped configuration of the valve. In some implementations, the distal end portion of the push shaft 220 can be flared radially outward, to terminate at a wider-diameter that can contact the prosthetic valve 260 in its crimped state. Push shaft 220 can then be advanced distally, pushing the prosthetic valve 260 therewith, until the crimped prosthetic valve 260 is disposed around the balloon 250, at which point the balloon 250 can be inflated to radially expand the prosthetic valve 260. Once the prosthetic valve 260 is expanded to its functional diameter within a native annulus, the balloon 250 can be deflated, and the delivery apparatus 200 can be retrieved from the patient's body.

In some examples, the delivery apparatus 200 with the prosthetic valve 260 assembled thereon, can be packaged in a sterile package that can be supplied to end users for storage and eventual use. In some examples, when the leaflets of the prosthetic valve are made from, or include at least an inner core made from, bovine pericardium tissue or other natural or synthetic tissues, the leaflets can be treated during the manufacturing process so that they are completely or substantially dehydrated and can be stored in a partially or fully crimped state without a hydrating fluid. In this manner, the package containing the prosthetic valve 260 and the delivery apparatus 200, can be free of any liquid. Methods for treating tissue leaflets for dry storage are disclosed in U.S. Pat. Nos. 8,007,992 and 8,357,387, both of which documents are incorporated herein by reference.

FIG. 4 shows a cross sectional view of prosthetic valve 10. In some cases, blood stagnation or low flow regions can occur between leaflets 14 and inner skirt 140, particularly at the lowest or distal-most regions along the scalloped line 106, in the vicinity of cusp edge midpoint 134. Such “pockets” of flow stagnation behind the leaflets can cause platelets to adhere to the outer surface 170 of the leaflet 14, until thrombus formation along the leaflet 14, such as between leaflet 14 and inner skirt 140. Since a leaflet polymeric outer surface 170 can resist tissue ingrowth, for example by being made of thromboresistant materials (such as, but not limited to, TPU) and/or by including a relatively smooth leaflet outer surface 170, adhesion of thrombotic formations 20 to the leaflet 14 is relatively loose, which is of critical concern because, if dislodged, such thrombus can create an embolus that may flow downstream and reach important systemic organs.

The term “thrombus” or “blood clot”, as used herein, refers to a solid or semi-solid mass that can include the constituents of blood that is the product of blood coagulation. There are two components to a thrombus, aggregated platelets that form a platelet plug, and a mesh of cross-linked fibrin protein.

FIG. 4 schematically illustrates several exemplary thrombus formations at various optional stages, with thrombus 20a shown to be formed over and loosely attached to the leaflet polymeric outer surface 170, for example within a “flow pocket” in the vicinity of cusp edge midpoint 134, and another thrombus 20b detached from the leaflet polymeric outer surface 170, free to migrate further downstream, posing a risk of eventually causing blood clots or blockage in other portions of the arteries.

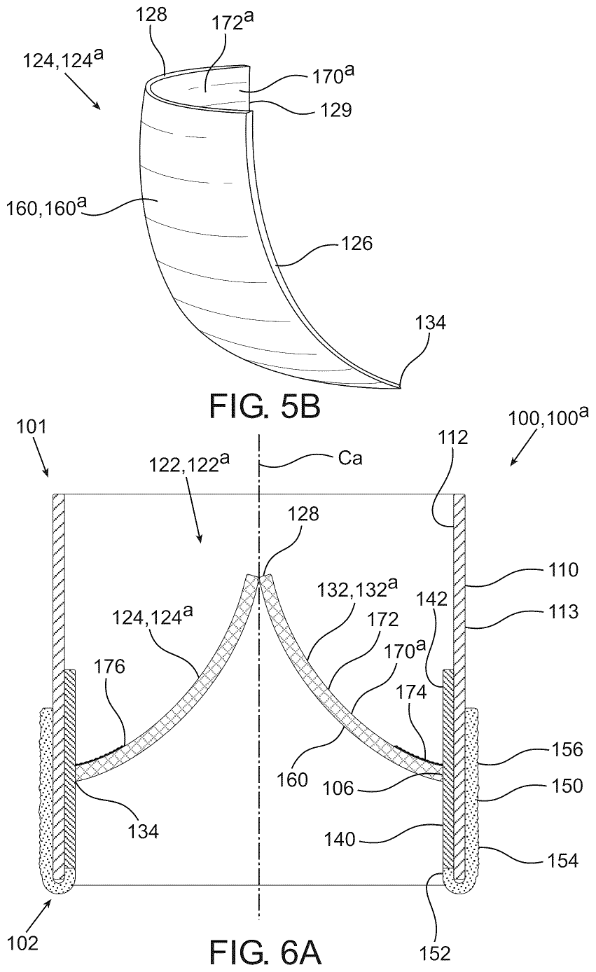

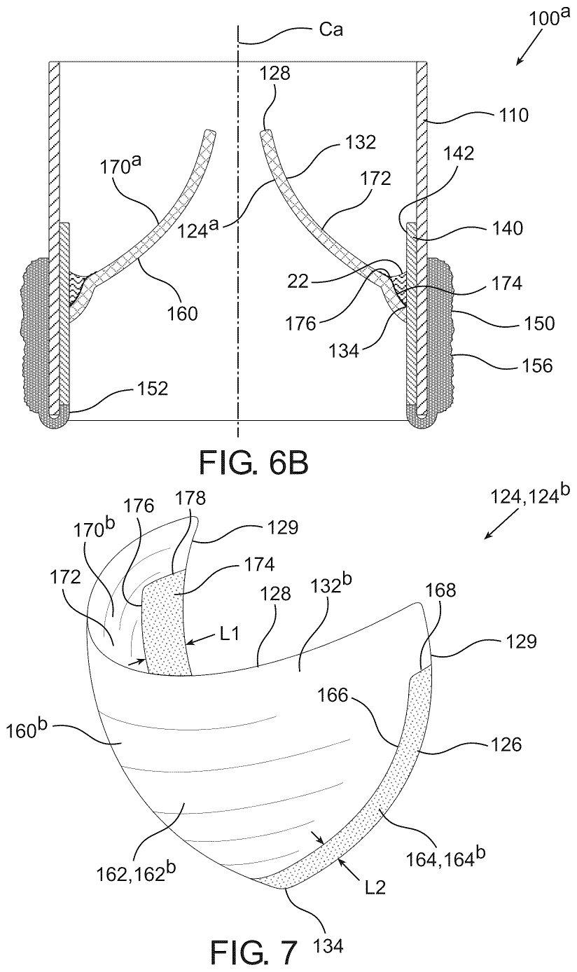

FIG. 5A-5B show views if perspective (from different angles of view) of an exemplary leaflet 124, which can be identical to any example described above for leaflet 14, except that leaflet 124 further comprises a leaflet polymeric outer surface 170 having at least a portion thereof configured to allow tissue ingrowth or tissue adherence thereto, also referred to as a tissue-adherent surface. FIG. 6A shows a cross-sectional view of an exemplary implementation of a prosthetic valve 100. Prosthetic valve 100 can be structurally and functionally similar to any example described above with respect to prosthetic valve 10, except that while prosthetic valve 10 includes a valvular structure 12 comprising leaflets 14 that can have a relatively uniform leaflet thromboresistant surface, such as at least along the outer surface 170 of the leaflet 124, prosthetic valve 100 includes a valvular structure 12, which is similar to any example described above with respect to valvular structure 12, except that the leaflets 124 of valvular structure 12 include at least a portion of at least one surface, such as outer surface 170 and/or inner surface 160, which is a tissue-adherent surface configured to allow and/or encourage tissue ingrowth or tissue adherence thereto. All other components and features of prosthetic valve 100, valvular structure 12, and leaflet 124, can be similar to the same components, with the same component numerals, and features thereof, described above with respect to prosthetic valve 10, valvular structure 12, and leaflet 14, and in the interest of brevity will not be described further.

The term “tissue-adherent surface”, as used herein, refers to a surface texture that causes or encourages adherence of tissue thereto. In some examples, a tissue adherent surface can be a thrombogenic surface, configured to cause or encourage development of a thrombus and coagulation of blood. In some examples, a tissue-adherent surface can be a surface that encourages formation of a neointimal tissue thereon. As exemplified herein, more textured or rough surfaces will have higher thrombogenicity and/or tissue adherence values compared to flatter smooth surfaces comprised of the same materials. Similarly two surfaces having the same structure may have different thrombogenicities and/or tissue adherence values depending on their chemical composition.

Relative thrombogenicity or tissue adherence values between articles that can include different materials, or include the same material but having different surface textures, can be determined, for example, according to the regulation (EU) 2017/745 of the European parliament and of the council on medical devices. The regulation refers to ISO10993-4-2017, and includes measurements to be performed in order to determine three main parameters relating to thrombogenicity: (1) thrombin generation, as measured by ELISA (enzyme-linked immunosorbent assay) for thrombin-antithrombin complex and prothrombin fragment F1+2; (2) fibrin generation as measured by ELISA for fibrinopeptide A; and (3) Intrinsic pathway (FXII) as measured by PTT (partial thromboplastin time) test.

The term “thromboresistant” refers to a material or an article (e.g., an implant or a part thereof), which is substantially resistant to biological damaging caused by platelet adhesion, thrombus formation and/or tissue ingrowth in vitro and/or in vivo. Specifically, “thromboresistant surface” refers to a surface texture which imparts thromboresistance to an article, such as a leaflet or a portion thereof. Some materials may be formed to have relatively smooth surfaces that resist tissue adherence or growth thereon, including resistance to adherence of thrombus or blood clots to the surface, and/or resistance to neointimal tissue overgrowth. Exemplary materials that can have thromboresistant surfaces include, but are not limited to, PTFE (polytetrafluoroethylene) and TPU (thermoplastic polyurethane).

The terms “leaflet outer surface” and “leaflet polymeric outer surface”, as used throughout the specification and the claims, are interchangeable, and refer either to a leaflet made from homogenous polymeric material that defines its surfaces, or a multi-layered leaflet coated by a polymeric layer that defines its outer surface 170.

The terms “leaflet inner surface” and “leaflet polymeric inner surface”, as used throughout the specification and the claims, are interchangeable, and refer either to a leaflet made from homogenous polymeric material that defines its surfaces, or a multi-layered leaflet coated by a polymeric layer that defines its inner surface 160.

Various exemplary implementations for prosthetic valves 100 and leaflets 124 thereof can be referred to, throughout the specification, with superscripts, for ease of explanation of features that refer to such exemplary implementations. It is to be understood, however, that any reference to structural or functional features of any apparatus, assembly or component, without a superscript, refer to these features being commonly shared by all specific exemplary implementations that can be also indicated by superscripts. In contrast, features emphasized with respect to an exemplary implementation of any apparatus, assembly or component, including prosthetic valves 100 and/or leaflets 124 thereof, referred to with a superscript, may be optionally shared by some but not necessarily all other exemplary implementations.

In some examples, a polymeric surface of a leaflet 124 can comprise at least one surface of a portion of a surface which is a tissue-adherent surface. In some examples, a polymeric surface of a leaflet 124 can comprise at least two surface portions that may have different textures. Surfaces or surface portions with different textures can include a tissue-adherent surface and a thromboresistant surface, or two tissue-adherent surfaces, one of which is a thrombogenic surface configured to encourage or allow thrombus formation and/or adherence thereto, while another is a neointimal-formation encouraging surface, configured to encourage neointimal tissue development thereover. In any example of a leaflet 124 that includes at least two differently textured surfaces or surface portions, the texture of one of the surfaces will be more thrombogenic than the other. In some examples, two differently textured surfaces are configured such that the thrombogenicity of one surface is at least 5% higher than that the other surface. In some examples, two differently textured surfaces are configured such that the thrombogenicity of one surface is at least 25% higher than that the other surface. In some examples, two differently textured surfaces are configured such that the thrombogenicity of one surface is at least 50% higher than that the other surface. In some examples, two differently textured surfaces are configured such that the thrombogenicity of one surface is at least 100% higher than that the other surface.

For example, a leaflet outer surface 170 can include a proximal outer surface portion 172 which is a thromboresistant surface, and a distal outer surface portion 174 which is a tissue-adherent surface. FIGS. 5A-5B show an exemplary leaflet 124^{a} having a leaflet outer surface 170^{a} that includes a proximal outer surface portion 172^{a} and a distal outer surface portion 174^{a}, wherein both portions 172^{a} and 174^{a} have different textures that influence the affinity of tissue adherence, including thrombus attachment, to each corresponding portion.

The distal outer surface portion 174 extends proximally from the cusp edge 126 to a proximal edge 176 of the distal outer surface portion 174, and can extend along the length of the cusp edge 126 between two proximal ends 178 of the distal outer surface portion 174. The proximal outer surface portion 172 can cover the remainder of the leaflet outer surface 170 which is not covered by the distal outer surface portion 174, and can extend proximally from the proximal edge 176 of the distal outer surface portion 174 to the free edge 128. The distal outer surface portion 174 can be in the shape of a strip, defining a textured outer portion height L1 between the cusp edge 126 and the proximal edge 176 of distal outer surface portion 174. The textured outer portion height L1 can be parallel to the central axis Ca at the position of cusp edge midpoint 134, and perpendicular to the cusp edge 126 at any other position. The height L0 of the proximal outer surface portion 172 (defined between the proximal edge 176 of portion 174 to the free edge 128) can be significantly greater than L1 at the position of cusp edge midpoint 134. In some examples, L0 is at least two times greater than L1. In some examples, L0 is at least three times greater than L1. In some examples, L0 is at least four times greater than L1.

Since a portion of the leaflet 124 is attached to the frame 110 (either directly or via intermediate components, such as inner skirt 140) along the scalloped line 106, textured outer portion height L1 can be measured, in some examples, as a positive value that extends along the leaflet body 132, which is the movable part of the leaflet 124 that excludes the immovable region of attachment.

In some examples, as shown in FIG. 5A, the textured outer portion height L1 can be uniform along its length between both ends 178, such that L1 at any position along the distal outer surface portion 174, including at the position of cusp edge midpoint 134 and proximal ends 178, is substantially identical. Stated otherwise, the proximal edge 176 can be substantially parallel to the cusp edge 126 in such examples.

In some examples, the distal outer surface portion 174 extends along most or all of the length of the cusp edge 126. In the example illustrated in FIG. 5A, the distal outer surface portion 174^{a} is shown to extend between the cusp edge midpoint 134 and the commissure attachment portions 129, such that its proximal ends 178 terminate at the border between the cusp edge 126 and the commissure attachment portions 129. It is to be understood that this is shown by way of illustration and not limitation, and that in some examples, the distal outer surface portion 174 can further extend towards the free edge 128, such that its proximal ends 178 can be positioned at any level along the commissure attachment portions 129, and in some examples the proximal ends 178 can be positioned at the free edge 128. Similarly, the distal outer surface portion 174 can be, in some examples, shorter, such that its proximal ends 178 are distal to the commissure attachment portions 129.

In some examples, one surface of the leaflet can include two separate portions having different textures, while the opposite surface can have a uniform texture. Exemplary leaflet 124^{a} has a leaflet outer surface 170^{a} that includes a proximal outer surface portion 172^{a} and a distal outer surface portion 174^{a} as described above and shown in FIG. 5A, while the leaflet inner surface 160^{a} can have a uniform texture as shown in FIG. 5B. In some examples, the leaflet inner surface 160^{a} can have a thromboresistant surface texture, similar to that described above with respect to proximal outer surface portion 172^{a}.

As shown in FIG. 6A, the leaflet 124^{a} can be attached to the inner skirt 140 along scalloped line 106, such that the distal outer surface portion 174^{a} extends from the scalloped line 106 along textured outer portion height L1. The distal outer surface portion 174, which can be also referred to as an outer thrombus anchoring portion 174 of the leaflet 124, is facing away from central axis Ca and towards the outflow end 101 of the valve and the skirt internal surface 142. The distal outer surface portion 174 is configured to encourage tissue ingrowth and/or tissue adherence thereto, such that any thrombus formed along the distal outer surface portion 174 will remain attached thereto, thereby reducing risk of thrombus dislodgment from the distal outer surface portion 174, such as in the vicinity of cusp edge midpoint 134 or other regions along the scalloped line 106. The distal outer surface portion 174 is configured to encourage tissue ingrowth thereover and/or tissue adherence thereto at a region bound between the leaflet outer surface 170 and an opposite surface such as skirt internal surface 142, such as due to low-flow, recirculation zone or stagnation pockets formed between the distal outer surface portion 174 and the inner skirt 140, above the scalloped line 106.

In some examples, the distal outer surface portion 174 is a tissue adherent surface, configured to encourage or allow tissue ingrowth or tissue adherence thereto. In some examples, the proximal outer surface portion 172 is a thromboresistant surface, configured to resist tissue adherence thereto. A tissue adherent surface can be also referred to as a textured surface, while a thromboresistant surface can be referred to as a smooth surface. The term “textured surface”, as used herein, refers to a surface having a topology with nano- to micron-sized surface variations formed by a texturing technique. While the characteristics of such a surface can be variable depending on the materials and techniques employed, such a surface can include micron-sized pores exposed to the external environment (such as blood in the vicinity of the textured surface), which have a depth that does not extend through the complete thickness of the leaflet 124.

As mentioned above, a leaflet 124 according to examples herein is made of, or comprises, a polymeric material, from which at least one surface thereof, such as a leaflet outer surface 170 and/or leaflet inner surface 160, is made. In some examples, the leaflet 124 is a polymeric leaflet, made of a polymeric material defining both surfaces 160 and 170. In some examples, any polymeric surface of a leaflet 124, and optionally the leaflet 124 as a whole, comprises a biocompatible polymer. In some examples, any polymeric surface of a leaflet 124, and optionally the leaflet 124 as a whole, consists of a biocompatible polymer. In some examples, any polymeric surface of a leaflet 124, and optionally the leaflet 124 as a whole, comprises a polyurethane. In some examples, any polymeric surface of a leaflet 124, and optionally the leaflet 124 as a whole, consists of a polyurethane. In some examples, any polymeric surface of a leaflet 124, and optionally the leaflet 124 as a whole, comprises thermoplastic polyurethane (TPU). In some examples, any polymeric surface of a leaflet 124, and optionally the leaflet 124 as a whole, consists of TPU.

The term “tissue adherence”, as used herein with respect to surface portions of leaflet 124, such as a distal outer surface portion 174, or an optional distal inner surface portion 164 which will be described in further detail below, refers both to tissue ingrowth directly within the corresponding surface portion or over the corresponding surface portion, as well as to the leaflet surface portion's ability to capture and maintain attachment with a thrombus that may be formed in close proximity thereto, such as within the “flow pocket” bound between the leaflet 124 and the frame 110 and/or inner skirt 140, or along an outer surface of the leaflet 124. For example, a thrombus that may initiate formation within the “flow pocket” due to stagnation or flow recirculation, may freely float within the pocket and be contacted by a textured surface (such as a porous surface) of the distal outer surface portion 174, upon which the thrombus will adhere to the distal outer surface portion 174, and remain attached thereto in a manner that prevents, or at least reduces the likelihood of, spontaneous dislodgment therefrom.

The distal outer surface portion 174 is configured to prevent spontaneous detachment of tissue adhered thereto, for a minimal period of time, after which spontaneous detachment or dislodgment becomes less likely. The term “spontaneous detachment”, as used herein, refers to detachment from the distal outer surface portion 174 when subjected to normal physiological and flow conditions at the site of implantation (or within an experimental setup that mimics such conditions). In some examples, the distal outer surface portion 174 is configured to prevent spontaneous detachment of tissue adhered thereto (either when implanted in a patient or in an experimental setup that mimics the flow conditions of a physiological implantation site) for a period of at least one month. In some examples, the distal outer surface portion 174 is configured to prevent spontaneous detachment of tissue adhered thereto for a period of at least six months. In some examples, the distal outer surface portion 174 is configured to prevent spontaneous detachment of tissue adhered thereto for a period of at least one year.

FIG. 6B shows a cross sectional view of the prosthetic valve 100^{a} of FIG. 6A with tissue ingrowth and/or thrombus adherence to the distal outer surface portion 174^{a}, representing a potential state of the valve 100^{a} after residing within the implantation site for a certain period of time. As mentioned above, the outer skirt 150, and more specifically, an outer portion 154 thereof, can be configured to allow tissue ingrowth in some examples, which may result in a bulkier or thicker formation of the outer skirt 150 over time, as schematically shown in FIG. 6B. Thrombus 22 that may have formed along the distal outer surface portion 174 of leaflet 124, is shown to remain firmly attached to the distal outer surface portion 174 due to the texture of the distal outer surface portion 174 configured to allow long-term adherence of tissue thereto, as described above.

In some cases, the portion of the skirt internal surface 142 facing the distal outer surface portion 174 of leaflet 124 and the thrombus 22 formed thereon, is also adhered to the opposite side of thrombus 22, such that the thrombus 22 is bound between, and attached to, both the internal surface 142 of inner skirt 140 and the distal outer surface portion 174 of leaflet 124. Attachment of thrombus 22 to the distal outer surface portion 174 of leaflet 124 may, in turn, inhibit motility of the part of the leaflet 124 attached to thrombus 22 to a certain extent, such that the portion of leaflet body 132 free to move between the open and closed states of the valvular structure 122 is the portion above thrombus 22.

FIG. 6A shows the valvular structure 122 in a closed state, wherein the leaflets 124 are shown to coapt with each other to prevent backflow through the main flow channel defined between the leaflets 124. The leaflets 124 can be dimensioned to allow their free edges 128 to move along the inner radius R (indicated, for example, in FIG. 4) between the open state, in which their free edges 128 can move closer to the inner surface 112 of the frame 110, and the closed state, moving toward each other and the central axis Ca. When a portion of the leaflet 124 is adhered to thrombus 22, such as along the distal outer surface portion 174, full coaptation may not be necessarily achievable. Nevertheless, it has been shown in a series of experiments that a limited degree of backflow may be allowed in the closed state, without adversely affecting the performance of the prosthetic valve.

In some cases, thrombus 22 is adhered to the distal outer surface portion 174 of leaflet 124 in a manner that can allow full coaptation between the leaflets 124, yet limit their opening, such that the resulting orifice area through which blood can flow between the leaflets in their open state is less than the orifice area in an un-constricted state.

Thus, the textured portion height L1 of distal outer surface portion 174 can be designed such that even when a portion of leaflet 124 adhered to a thrombus 22 along distal outer surface portion 174 extending from the scalloped line 106, optionally from the cusp edge midpoint 134, along a length equal to the length L1, the amount of backflow through the valvular structure 122 will still not exceed the tolerable threshold beyond which the performance of the valve 100 can be adversely affected, and/or the flow of blood through a potentially narrower orifice area between the leaflets will still be sufficient to maintain adequate valve functioning. In some examples, the textured portion height L1 is equal to or less than 20% of internal radius R of prosthetic valve 100 in the expanded state. In some examples, the textured portion height L1 is equal to or less than 10% of internal radius R of prosthetic valve 100 in the expanded state. In some implementations, a distal outer surface portion 174 can have a non-uniform textured portion height L1 between the position of the cusp edge midpoint 134 and the proximal ends 178, as will be described in greater detail with respect to FIG. 10 herein. In such cases, any reference to a textured portion height L1 not exceeding a certain percentage of the radius R can refer to a maximal textured portion height L1, which can be at the position of cusp edge midpoint 134. In some examples, the textured outer portion height L1 is between about 1 millimeter (mm) and about 3 mm, including each value and sub-range within this range.

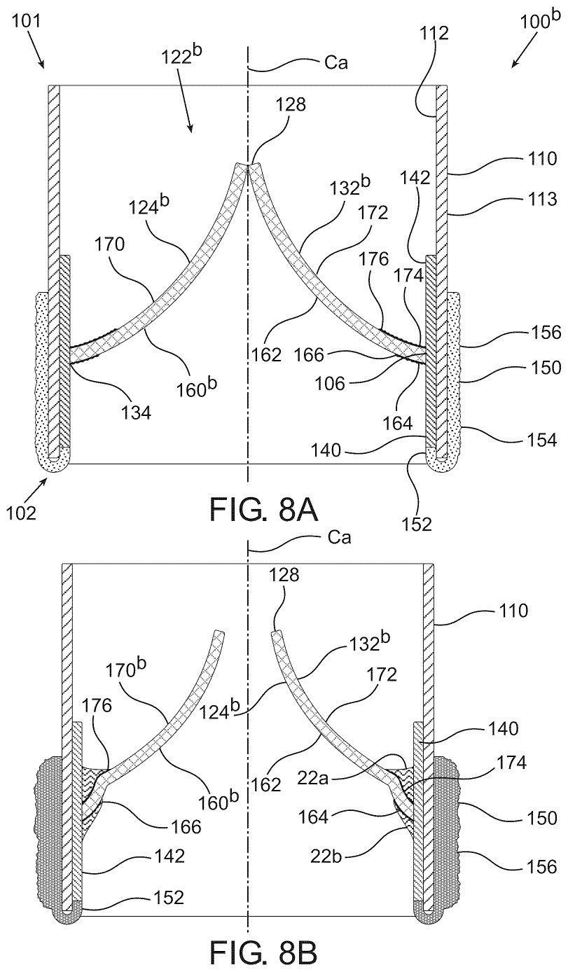

In some examples, the leaflet inner surface 160 comprises a proximal inner surface portion 162 and a distal inner surface portion 164, in addition to, or in some examples, instead of, the proximal outer surface portion 172 and a distal outer surface portion 174 of the leaflet outer surface 170. FIG. 7 shows a view in perspective of an exemplary leaflet 124^{b}. Leaflet 124^{b} can be similar to any example described above with respect to exemplary leaflet 124^{a}, including a leaflet polymeric outer surface 170^{b} that comprises a proximal outer surface portion 172 and a distal outer surface portion 174, except that while leaflet 124^{a} may have a uniformly textured leaflet inner surface 160^{a}, the leaflet inner surface 160^{b} of leaflet 124^{b} comprises a proximal inner surface portion 162^{b} and a distal inner surface portion 164^{b}. FIG. 8A shows a cross-sectional view of an exemplary prosthetic valve 100^{b} comprising a valvular structure 122^{b} that includes leaflets 124^{b} of the type shown in FIG. 7. The proximal inner surface portion 162 can be generally similar to proximal outer surface portion 172, implemented as a thromboresistant surface, and the distal inner surface portion 164 can be generally similar to distal outer surface portion 174, implemented as a tissue-adherent surface.

The distal inner surface portion 164 extends proximally from the cusp edge 126 to a proximal edge 166 of the distal inner surface portion 164, and can extend along the length of the cusp edge 126 between two proximal ends 168 of the distal inner surface portion 164. The proximal inner surface portion 162 can cover the remainder of the leaflet inner surface 160 which is not covered by the distal inner surface portion 164, and can extend proximally from the proximal edge 166 of the distal inner surface portion 164 to the free edge 128. The distal inner surface portion 164 can be in the shape of a strip, defining a textured inner portion height L2 between the cusp edge 126 and the proximal edge 166 of distal inner surface portion 164. The textured inner portion height L2 can be parallel to the central axis Ca at the position of cusp edge midpoint 134, and perpendicular to the cusp edge 126 at any other position.

Since a portion of the leaflet 124 is attached to the frame 110 (either directly or via intermediate components, such as inner skirt 140) along the scalloped line 106, textured inner portion height L2 can be measured, in some examples, as a positive value that extends along the movable leaflet body 132, excluding the immovable region of attachment.

In some examples, as shown in FIG. 7, the textured inner portion height L2 can be uniform along its length between both ends 168, such that L2 at any position along the distal inner surface portion 164, including at the position of cusp edge midpoint 134 and proximal ends 168, is substantially identical. Stated otherwise, the proximal edge 166 can be substantially parallel to the cusp edge 126 in such examples. In some examples, the textured inner portion height L2 is between about 1 mm and about 3 mm, including each value and sub-range within this range.

In some examples, the distal inner surface portion 164 extends along most or all of the length of the cusp edge 126. In the example illustrated in FIG. 7, the distal inner surface portion 164^{b} is shown to extend between the cusp edge midpoint 134 and the commissure attachment portions 129, such that its proximal ends 168 terminate at the border between the cusp edge 126 and the commissure attachment portions 129. It is to be understood that this is shown by way of illustration and not limitation, and that in some examples, the distal inner surface portion 164 can further extend towards the free edge 128, such that its proximal ends 168 can be positioned at any level along the commissure attachment portions 129, and in some examples the proximal ends 168 can be positioned at the free edge 128. Similarly, the distal inner surface portion 164 can be, in some examples, shorter, such that its proximal ends 168 are distal to the commissure attachment portions 129.

Similar to distal outer surface portion 174, the distal inner surface portion 164 can be configured to prevent spontaneous detachment of tissue adhered thereto, for a minimal period of time, after which spontaneous detachment or dislodgment becomes less likely. In some examples, the distal inner surface portion 164 is configured to prevent spontaneous detachment of tissue adhered thereto (either when implanted in a patient or in an experimental setup that mimics the flow conditions of a physiological implantation site) for a period of at least one month. In some examples, the distal inner surface portion 164 is configured to prevent spontaneous detachment of tissue adhered thereto for a period of at least six months. In some examples, the distal inner surface portion 164 is configured to prevent spontaneous detachment of tissue adhered thereto for a period of at least one year.

As shown in FIG. 8A, the leaflet 124^{b} can be attached to the inner skirt 140 along scalloped line 106, such that the distal inner surface portion 164^{b} extends from the scalloped line 106 along textured inner portion height L2. The distal inner surface portion 164, which can be also referred to as an inner thrombus anchoring portion 164 of the leaflet 124, is facing towards the central axis Ca and the inflow end 102 of the valve, and away from the skirt internal surface 142. The distal inner surface portion 164 is configured to encourage tissue ingrowth and/or tissue adherence thereto, such that any thrombus formed along the distal inner surface portion 164 will remain attached thereto, thereby reducing risk of thrombus dislodgment from the distal inner surface portion 164, such as in the vicinity of cusp edge midpoint 134 or other regions along the scalloped line 106. The distal inner surface portion 164 is configured to encourage tissue ingrowth thereover and/or tissue adherence thereto at a region oriented towards the inflow end 102 of the valve 100, below the scalloped line 106. While flow disturbances that can encourage thrombosis are more prominent within the “flow pockets” in the vicinity of cusp edge midpoints 134 above the scalloped line 106, flow disturbances can occasionally occur also below the leaflets 124 in the vicinity of and distal to the scalloped line 106 and/or cusp edge midpoints 134, optionally to a lesser extent, yet sufficient to encourage thrombosis in such regions as well, in which case it may be of advantage for the leaflet 124 to include a distal inner surface portion 164 in addition to the distal outer surface portion 174.

When leaflet 124^{b} includes both a distal outer surface portion 174 and a distal inner surface portion 164, their heights can be similar or different from each other. In some examples, the textured outer portion height L1 can be substantially equal to textured inner portion height L2 (example not illustrated). The term substantially equal to, as used herein, refer to being within a range of ±10% of the target value. In some examples, the textured outer portion height L1 can be greater than the textured inner portion height L2, as illustrated in FIG. 7. In some examples, the textured outer portion height L1 is at least twice as great as the textured inner portion height L2 (e.g., L1≥2L2).

FIG. 8B shows a cross sectional view of the prosthetic valve 100^{b} of FIG. 8A with tissue ingrowth and/or thrombus adherence to different portions thereof, representing a potential state of the valve 100^{b} after residing within the implantation site for a certain period of time. Thrombus 22^{a} that may have formed along the distal outer surface portion 174 of leaflet 124, is shown to remain firmly attached to the leaflet 124 due to the pro-thrombotic texture provided by the distal outer surface portion 174 to allow long-term adherence of tissue thereto, as described above. Another thrombus 22^{b} is shown in FIG. 8B formed along the distal inner surface portion 164 of the leaflet, similarly demonstrated to remain attached to the leaflet 124 with reduced risk of dislodging therefrom.

In some examples, the proximal outer surface portion 172 and the distal outer surface portion 174 are made from the same polymeric material, but manufactured or treated to have different textures in each portion. In some examples, the proximal inner surface portion 162 and the distal inner surface portion 164 are made from the same polymeric material, but manufactured or treated to have different textures in each portion. In some examples, the leaflet outer surface 170, including proximal 172 and distal 174 portions thereof, and the leaflet inner surface 160, including proximal 162 and distal 164 portions thereof, are made from the same polymeric material. In some examples, the proximal outer surface portion 172 and the proximal inner surface portion 162 have similar textures, configured to be relatively thromboresistant. In some examples, the distal outer surface portion 174 and the distal inner surface portion 164 have similar textures, configured to be relatively pro-thrombotic.

In some examples, the leaflet 124 is made from a single homogenous polymeric material, such that all surfaces thereof, including leaflet outer surface 170 and any portion thereof, and leaflet inner surface 160 and any portion thereof, are similarly made from the same polymeric material, optionally manufactured or treated to have different textures along different surface portions of the leaflet.

In some examples, as mentioned above, a leaflet 124 having polymeric inner 160 and outer 170 surfaces, can be provided as a coated or multi-layered leaflet. FIG. 9 shows an exemplary leaflet 124^{c}. Leaflet 124^{c} is an exemplary implementation of leaflet 124, and thus includes all of the features described for leaflet 124 throughout the current disclosure, except that leaflet 124^{c} is a multi-layered leaflet that includes a base layer 144 and at least an outer layer 148 that comprises a polymeric material, defining the leaflet polymeric outer surface 170^{c}. In some examples, leaflet 124^{c} further comprises an inner layer 146 that comprises a polymeric material, defining the leaflet polymeric inner surface 160^{c}. While the base layer 144 is illustrated in FIG. 9 to be coated on both sides by layers 148 and 146, it is to be understood that in some examples, the base layer 144 can be coated only on one side. For example, a coated or multi-layered leaflet 124^{c} can include a base layer 144 coated by an outer polymeric layer 148, but devoid of an inner layer, such that while the outer layer 148 defines the leaflet polymeric outer surface 170^{c}, the opposite surface of the base layer 144 can serve as a leaflet inner surface, which may or may not be a polymeric surface. It is to be understood that leaflet 124^{c} and layers thereof are not shown to scale, and that thicknesses of such layers can be significantly thinner in practice than the illustrated example.

In some examples, when including both an outer layer 148 and an inner layer 146, both outer and inner layers 148 and 146 can be made of the same polymeric material. In some examples, each of the outer layer 148 and the inner layer 146 can comprise a different polymeric material. The leaflet thickness T_{L} of leaflet 124^{c} can include the combined thickness of all two or three layers of the leaflets, such as the combined thickness of outer layer 148, base layer 144, and when present, inner layer 146.

The base layer 144 can be made of any material, including a polymeric material (which can be similar to, or different from, the polymeric material of any of the outer layer 148 and/or inner layer 146), fabric, or tissue. In some cases, it may be desired to utilize polymeric materials for forming surfaces of a leaflet, as polymers can advantageously allow for better control of surface textures, enabling improved control over desired thromboresistant and/or pro-thrombotic zones of the leaflet. However, some polymers cannot be used as the sole material forming the leaflets, as they may lack the material properties required to provide adequate mechanical performance. In such cases, a base layer 144, which can be made from tissue or fabric, as well as specific types of relatively rigorous polymeric materials, can be used to provide the desired mechanical performance (including, for example, a desired tear resistance), while the coated layers 146, 148 can provide the desired surface textures.

The leaflet outer surface 170^{c} defined by outer layer 148 can include a proximal outer surface portion 172 and a distal outer surface portion 174, following any of the examples described herein for such portions. When an inner layer 146 is provided, the leaflet inner surface 160^{c} defined thereby (hidden from view in FIG. 9) can similarly include a proximal inner surface portion 162 and a distal inner surface portion 164, following any of the examples described herein for such portions.

Any of the heights L1 or L2 can be uniform or non-uniform along the circumference of the leaflet, and particularly between the position of the cusp edge midpoint 134 and the respective proximal ends 178 or 168, respectively. In some examples, the textured outer portion height L1 can be greater at the position of cusp edge midpoint 134 and narrower at the proximal ends 178. Such configurations can be advantageous when flow stagnation or other flow disturbances are mostly prominent in the distal-most “pockets” formed between the leaflets 124 and the frame 110 (including between leaflet 124 and inner skirt 140 or any other components disposed around the inner surface 112 of the frame), in the vicinity of cusp edge midpoints 134, increasing the likelihood of thrombosis in this regions, compared with the likelihood of thrombus formation at more proximal regions closer to the outflow end 101, or the expected size of thrombotic tissue formed closer to the proximal ends 178 of the distal outflow surface portion 174.

FIG. 10 shows a flattened view of an exemplary leaflet 1244, with the distal outer surface portion 174 and its proximal edge 176 shown in full lines, while the distal inner surface portion 164 and its proximal edge 166 are shown with dashed lines. Leaflet 124d is an exemplary implementation of leaflet 124, and thus includes all of the features described for leaflet 124 throughout the current disclosure, except that at least one of the distal outer surface portion 174 and/or distal inner surface portion 164 has a non-uniform height along the circumference of the leaflet. The term “circumference of the leaflet”, as used herein, refers to a path following the contour of the cusp edge 126, which can extend towards the commissure attachment portions 129 or even further towards the free edge 128.

The heights L1_{D} and L1_{P} denote the textured outer portion height at the position of cusp edge midpoint 134 and the proximal ends 178 of distal outer surface portion 174, respectively. The heights L2_{D} and L2_{P} denote the textured inner portion height at the position of cusp edge midpoint 134 and the proximal ends 168 of distal inner surface portion 164, respectively. In the illustrated example, the distal outer surface portion 174 is shown to be narrowing from a greater height L1_{D} at the position of the cusp edge midpoint 134 to a smaller height L1_{P} at the proximal ends 178 (e.g., L1_{D}>L1_{P}), and the distal inner surface portion 164 is shown to be narrowing from a greater height L2_{D} at the position of the cusp edge midpoint 134 to a smaller height L2_{P} at the proximal ends 168 (e.g., L2_{D}>L2_{P}). The heights L1_{D} and L2_{D} can be equal to each other, or different from each other. In some examples, the height L1_{D} is at least two times as great as the height L2_{D}. In some examples, the height L1_{D} is between about 1 mm and about 3 mm, including each value and sub-range within this range. In some examples, the height L2_{D} is between about 1 mm and about 3 mm, including each value and sub-range within this range.

While both the distal outer surface portion 174 and distal inner surface portion 164 are shown in FIG. 10 to have non-uniform textured portion heights L1 and L2, respectively, it is to be understood that this is shown by way of illustration and not limitation. In some examples, the distal outer surface portion 174 can have a non-uniform textured outer portion height L1, for example such as that illustrated in FIG. 10, while the distal inner surface portion 164 can have a uniform textured inner portion height L2 (e.g., L2_{D}=L2_{P}), as illustrated for example in FIG. 7.

In some implementations, the outer skirt 150 is configured to be bioresorbable. A bioresorbable outer skirt 150 may be constructed of a bioresorbable fabric or other bioresorbable materials. Upon implantation, the bioresorbable outer skirt 150 may be configured to dissolve, and may be removed due to the dissolving and/or may be replaced by tissue in its place. Such a feature may result in tissue formation around the implanted prosthetic valve 100 that may enhance sealing against the surrounding anatomy.

In some implementations, the inner skirt 140 is configured to be relatively thromboresistant. For example, the inner skirt 140 can include at least one or more of a polytetrafluoroethylene (PTFE), an ultra-high molecular weight polyethylene (UHMWPE), or a coated thermoplastic polyurethane (TPU). The polytetrafluoroethylene (PTFE) may comprise expanded polytetrafluoroethylene (ePTFE) in some examples as desired. Various other thromboresistant materials may be utilized as desired. In some examples, a thromboresistant coating may be utilized with the inner skirt 140. For example, a coating of PTFE or ePTFE may be applied to material of the inner skirt 140, which may comprise polyethylene terephthalate (PET) or another form of material. The underlying material of the inner skirt 140 in such examples, may be a material that allows for tissue ingrowth, such as PET, yet is coated with a thromboresistant coating. In some examples, the material of the inner skirt 140 may be fabricated to be thromboresistant. For example, a knit pattern of the material of the inner skirt 140 may be configured to be thromboresistant, with micropatterns or other forms of knit patterns. A knit PTFE fabric may be utilized in some examples. The material of the inner skirt 140 can include a smooth texture to be thromboresistant and inhibit tissue growth. Combinations of such features may be utilized to result in a thromboresistant inner skirt 140.

In the case of a thromboresistant inner skirt 140 designed to resist tissue ingrowth, adhesion of thrombotic formations to the inner skirt 140, opposite to the distal outer surface portion 174 of the leaflet 124, will be relatively loose. It may be desirable to encourage thrombus adhesion noy only to the distal outer surface portion 174, but also to a component of the prosthetic valve on the opposite side of the thrombus, facing the leaflet. In some examples, the prosthetic valve 100 further comprises a strip disposed internally to the frame.

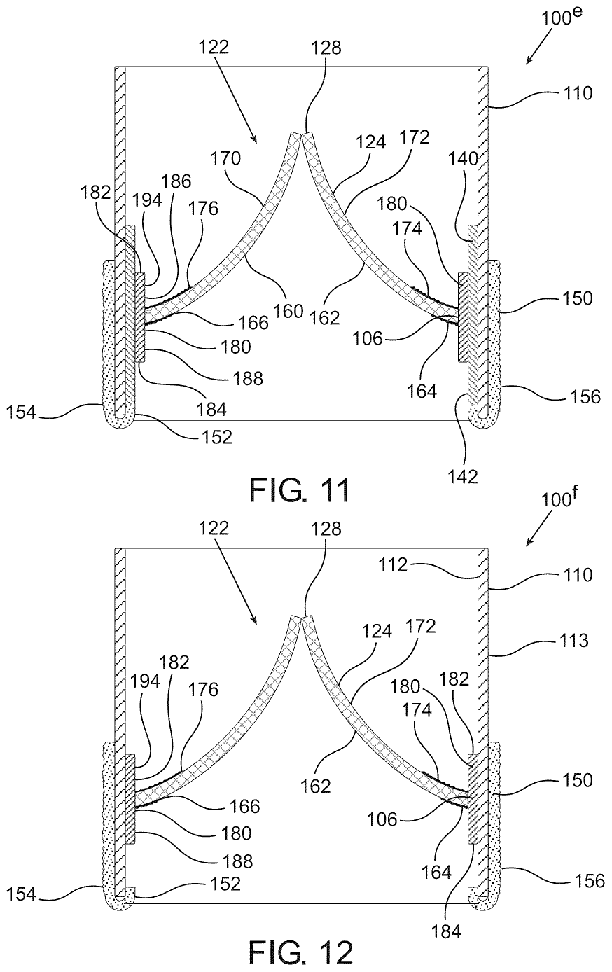

FIG. 11 shows a cross-sectional view of an exemplary prosthetic valve 100^{e}. Prosthetic valve 100^{e} is an exemplary implementation of prosthetic valve 100, and thus includes all of the features described for prosthetic valve 100 throughout the current disclosure, except that prosthetic valve 100^{e} further comprises a strip 180 disposed around and attached to the skirt internal surface 142, while the leaflets 124 are attached along the scalloped line 106 to the strip 180, such that the leaflets 124 extend inward (e.g., toward central axis Ca) from an internal surface 194 of the strip 180.

The strip 180 can be disposed around the entire circumference of inner skirt 140, and more particularly, the internal surface 142 of inner skirt 140. The strip 180 can be coupled to the inner skirt 140 via bonding, stitching, or another form of coupling. The strip 180 comprises a proximal edge 182 and a distal edge 184. The strip's height, defined between its proximal 182 and distal 184 edges, can be less than the height of the inner skirt 140 (heights of the strip and inner skirt are not annotated separately), such that the proximal edge 182 of the strip 180 can be distal to the proximal edge of the inner skirt 140, and such that the distal edge 184 of the strip 180 can be proximal to the distal edge of the inner skirt 140.

The leaflets 124 are attached to the strip 180 along scalloped line 106 at a position distal to the proximal edge 182 of the strip 180, such that the strip 180 comprises a proximal portion 186 extending between the scalloped line 106 and the proximal edge 182 of the strip. In some examples, the scalloped line 106 is positioned between the proximal edge 182 and the distal edge 184 of the strip, and in particular, proximal to the distal edge 184, such that the strip 180 further comprises a distal portion 188 extending between the scalloped line 106 and the distal edge 184 of the strip. While an exemplary strip 180 is shown in the illustrated examples to include both a proximal portion 186 and a distal portion 188, it is to be understood that this is not mandatory, and that in some examples, the strip 180 does not include a distal portion 188, such that the scalloped line 106 is formed along the distal edge 184 of the strip (example not illustrated).

The strip 180, which can be also referred to as a thrombus anchoring strip 180, comprises a strip internal surface 194, defined as the surface facing toward central axis Ca and away from frame 110. The strip 180, and in particular its internal surface 194, is configured to encourage tissue ingrowth and/or tissue adherence thereto, such that any thrombus formed along the strip 180 will remain attached thereto, This can be in addition to the leaflet 124 comprising one or more tissue-adherent surfaces, such that a thrombus can be attached both to the strip 180 on one side, and to a corresponding tissue-adherent surface of the leaflet 124 on another side, thereby reducing risk of thrombus dislodgment from the strip 180 and the leaflet 124, such as in the vicinity of cusp edge midpoint 134 or other regions along the scalloped line 106. The proximal portion 186 is configured to encourage tissue ingrowth thereover and/or tissue adherence thereto at a region bound between a distal outer surface portion 174 of the leaflet 124 and the strip internal surface 194 along the strip's proximal portion 186.

The valvular structure 122 of prosthetic valve 100^{e} can include any of the exemplary leaflet 124 disclosed herein. For example, while a valvular structure 122 is shown in FIG. 11 to include leaflets 124 having both distal outer surface portion 174 and distal inner surface portion 164, which can be implemented in a manner similar to that described above with respect to leaflet 124^{b}, in some examples, the leaflets 124 of prosthetic valve 100^{e} can include distal outer surface portion 174 without a distal inner surface portion 164, optionally implemented in a manner similar to that described above with respect to leaflet 124^{a}.

When present, the distal portion 188 of the strip 180 can be configured to similarly encourage tissue ingrowth thereover and/or tissue adherence thereto at a region below the scalloped line 106. When the leaflet 124 does include a distal inner surface portion 164, a thrombus can be attached both to the distal inner surface portion 164 of the leaflet 124 and the strip internal surface 194 along the strip's distal portion 188.

In some examples, the strip 180 is configured to allow tissue ingrowth or tissue adherence thereto. In some examples, the strip 180 can include polyethylene terephthalate (PET) or another form of material that is configured to allow tissue ingrowth or tissue adherence thereto. In some examples, a coating may be utilized with the strip 180 that is configured to allow tissue ingrowth or tissue adherence thereto. For example, a porous coating or other form of coating may be applied to a thromboresistant material such as ultra-high molecular weight polyethylene (UHMWPE) to allow for tissue ingrowth or tissue adherence thereto. The underlying material of the strip 180 in such examples, may be a material that inhibits tissue ingrowth, yet is coated with a coating that allows tissue ingrowth or tissue adherence thereto, at least along the proximal portion 186. In some examples, the strip internal surface 194 is a tissue-adherent surface. In some examples, the material of the strip 180 may be fabricated to allow tissue ingrowth or tissue adherence thereto. For example, a knit pattern of the material of the strip 180 may be configured to allow tissue ingrowth or adherence, with a large knit pattern or other forms of knit patterns formed along the strip internal surface 194. A knit PET fabric may be utilized in some examples. In some examples, the strip 180 may include yarns (e.g., textured yarns) extending radially inward. The material of the strip 180 may include a porous texture to allow for tissue ingrowth. Combinations of such features may be utilized to result in a strip 180 that allows for tissue ingrowth or tissue adherence thereto.

In some examples, prosthetic valve 100 can be devoid of an inner skirt 140, yet include a strip 180. FIG. 12 shows a cross-sectional view of an exemplary prosthetic valve 100^{f}. Prosthetic valve 100^{f} can be similar to any example described above with respect to prosthetic valve 100^{e}, except that prosthetic valve 100^{f} is devoid of an inner skirt 140, such that the strip 180, which can be implemented according to any example described above, is disposed over, and is in contact with, the frame inner surface 112. The strip 180 can be directly attached to the frame 110, such as being sutured or otherwise coupled to struts 114 of the frame 110, and/or attached to the outer skirt 150, such as by sutures or other types of couplers that can extend through openings in cells 120 of the frame 110.

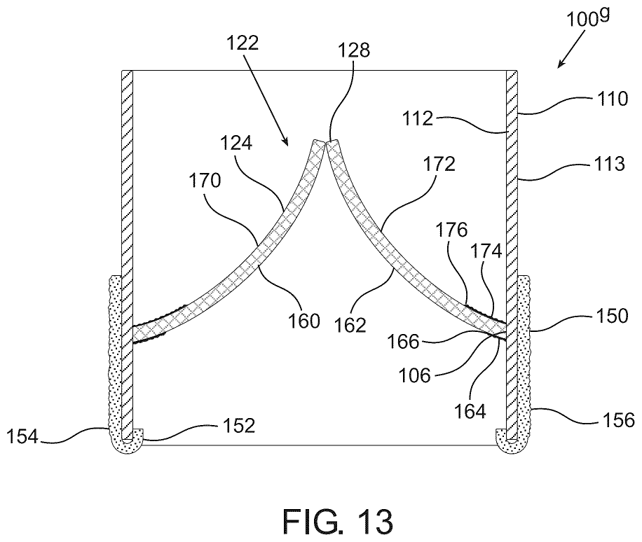

In some examples, prosthetic valve 100 can be devoid of both an inner skirt 140 and a strip 180. FIG. 13 shows a cross-sectional view of an exemplary prosthetic valve 100^{g}. Prosthetic valve 100^{g} is an exemplary implementation of prosthetic valve 100, and thus includes all of the features described for prosthetic valve 100 throughout the current disclosure, except that prosthetic valve 100^{g} is devoid of both an inner skirt 140 and a strip 180, such that the leaflets 124 are attached to the frame 110 along the scalloped line 106, such as by being coupled to struts 114 of the frame 110 and/or coupled to the outer skirt 150, optionally through openings in cells 120 of the frame 110.

The valvular structure 122 of prosthetic valve 100^{g} can include any of the exemplary leaflet 124 disclosed herein. For example, while a valvular structure 122 is shown in FIG. 13 to include leaflets 124 having both distal outer surface portion 174 and distal inner surface portion 164, which can be implemented in a manner similar to that described above with respect to leaflet 124^{b}, in some examples, the leaflets 124 of prosthetic valve 100^{g} can include distal outer surface portion 174 without a distal inner surface portion 164, optionally implemented in a manner similar to that described above with respect to leaflet 124^{a}.

In some examples, a leaflet 124 can include a tissue-adherent surface or surface portion which is not a thrombogenic surface, but is rather configured to allow neointimal tissue formation thereover, also referred to a neointimal-formation encouraging surface. Any type of a tissue-adherent surface disclosed herein can be implemented as a porous polymeric surface, except that the pore size of a thromboresistant surface can be generally greater than the pore size of a neointimal-formation encouraging surface.

The term “neointimal tissue”, also referred to herein as a tissue layer 30 (shown in FIG. 15) that can form along one or more surfaces or surface portions of leaflet 124, refers to a thin layer of tissue having an average thickness (in a direction perpendicular to the surface) of no more than 200 μm (or another average thickness threshold). The neointimal-formation encouraging surface can have an adequate degree of porosity configured to encourage ingrowth and/or adherence of a neointimal tissue layer that does not exceed the desired thickness. Thus, it is to be understood that when a tissue-adherent surface is implemented as a neointimal-formation encouraging surface, configured to allow or encourage formation of a tissue layer that has an average thickness of 200 μm (or another average thickness threshold), it differs from a tissue-adherent surface implemented as a thrombogenic surface, which is configured to allow adherence of thrombus or other tissue layers of any thickness, including thrombotic formations having an average thickness exceeding 200 μm (or another average thickness threshold), or even exceeding 0.5 mm.

In some examples, a polymeric surface of a leaflet 124, such as leaflet inner surface 160 and/or leaflet outer surface 170, can include at least two surface portions-both of which are tissue-adherent surfaces, but with differently dimensioned pores, such that the pores of one surface portion, implemented as a thrombogenic surface, can be relatively larger to allow adherence of thrombus of any size thereto, while the pores of another surface portion, implemented as a neointimal-formation encouraging surface, can be relatively smaller to encourage neointimal tissue formation that will not exceed an average thickness of about 200 μm (or another average thickness threshold).

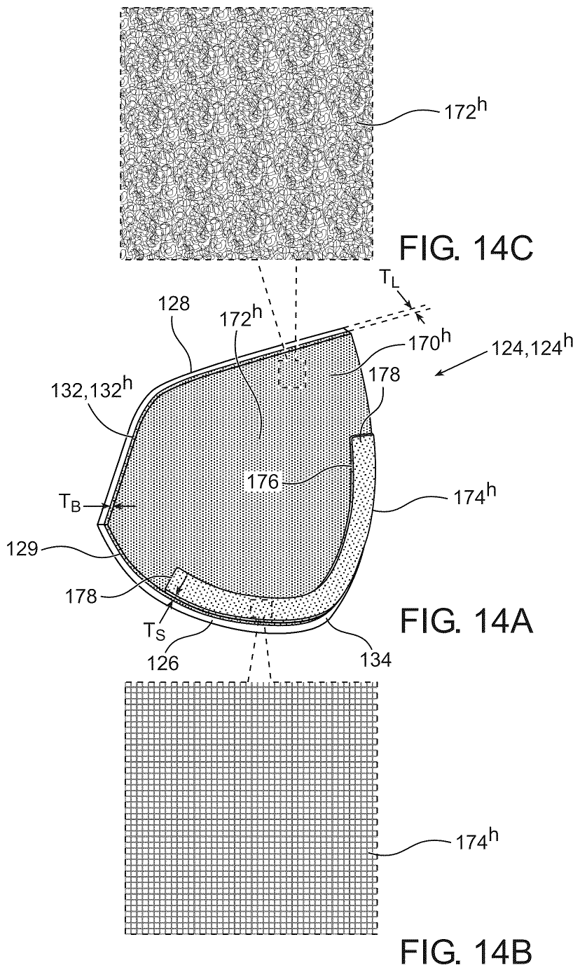

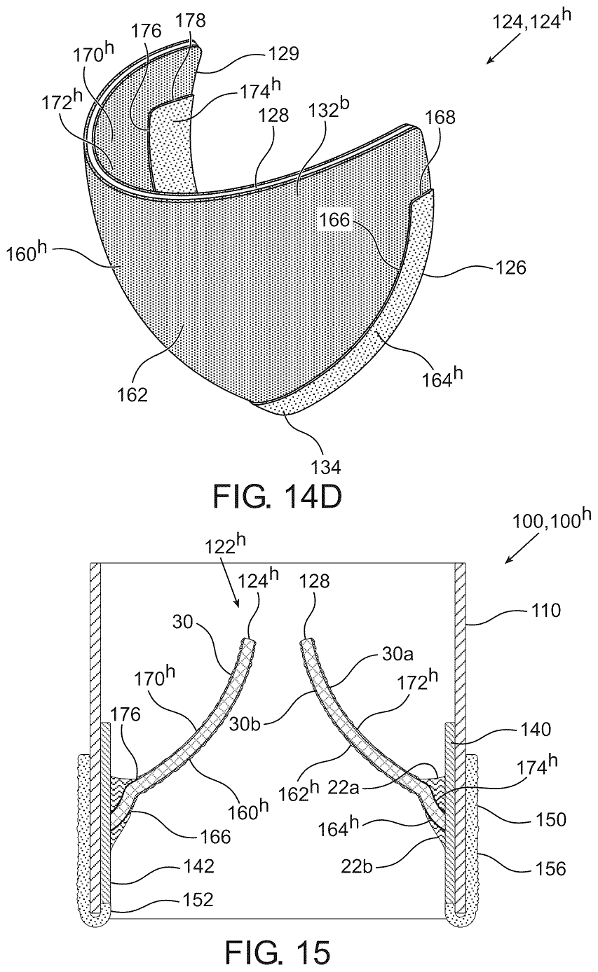

FIG. 14A shows a view in perspective of an exemplary leaflet 124^{h}. Leaflet 124^{h} is an exemplary implementation of leaflet 124, and thus includes all of the features described for leaflet 124 throughout the current disclosure, except that at least one surface of leaflet 124^{h} includes both a thrombogenic surface portion and a neointimal-formation encouraging surface portion. In the illustrated example, the leaflet outer surface 170^{h} is shown to include a proximal outer surface portion 172^{h} implemented as a neointimal-formation encouraging surface, and a distal outer surface portion 174^{h} implemented as a thrombogenic surface. FIGS. 14B and 14C are enlarged views of regions of the distal outer surface portion 174^{h} and the proximal outer surface portion 172^{h} of FIG. 14A, respectively, schematically showing exemplary surface textures of a thrombogenic surface (FIG. 14B) and a neointimal formation encouraging surface (FIG. 14C) of the corresponding portions.

In some examples, a tissue-adherent surface of a leaflet 124 is formed by electrospinning. In general, electrospinning is a method that produces fine fibers by charging and ejecting a polymer melt or solution through a spinneret under a high-voltage electric field, wherein the melt or solution is then solidified or coagulated to form a filament. The formed filaments are ultrafine, which can be in the hundreds of nanometers range in some examples. During the electrospinning process, electric force is applied to the polymer solution or melt to draw charged threads therefrom. When a sufficiently high voltage is applied to the droplet of the polymer melt or solution, the body of the liquid becomes charged, and electrostatic repulsion counteracts the surface tension and the droplet is stretched. At a critical point a stream of liquid erupts from the surface to form a charged liquid jet. As the jet dries in flight and deposited on a surface, the ultrafine filament is formed.

In some examples, formation of a tissue-adherent surface of a leaflet 124 comprises electrospraying a polymer over a polymeric surface of the leaflet 124 to form a textured layer thereon. In some examples, formation of a tissue-adherent surface of a leaflet 124 comprises providing a liquid polymer composition contained in an electrospray device and electrospraying the liquid polymer composition over a polymeric surface of the leaflet 124 to form a textured layer thereon. The electrospray device can include a container and a spinneret, in fluid communication therewith, the liquid polymer composition is contained in the container at the beginning of a procedure for formation of the tissue-adherent surface.

In some examples, the liquid composition comprises a melt polymer. The terms “melt polymer” and “polymer melt” are interchangeable and refer to a polymer which exist in a condense pliable form. It is to be understood that polymers may exist in pliable form either as liquids or in a state above their glass transition temperature. It is further to be understood that dilute solutions (e.g., having polymer content below 20% w/w) are not included under the terms “melt polymer” and “polymer melt”, as these are not condensed phases of the polymer.

In some examples, the liquid composition comprises a solution, suspension or emulsion containing the polymer. In some examples, the solution, suspension or emulsion contains the polymer at a concentration of 0.1% to 1% w/w, 1% to 2% w/w, 2% to 5% w/w, 5% to 10% w/w, 10% to 25% w/w or 25% to 50% w/w. In some examples, the liquid composition comprises a solution of the polymer. In some examples, the solution comprises a non-aqueous solvent. In some examples, the solvent is an organic solvent. In some examples, the solvent is selected from the group consisting of: dimethylformamide (DMF), dimethylacetamide (DMA), tetrahydrofuran (THF) ethyl acetate, and combinations thereof. In some examples, the solvent comprises dimethylformamide.

In some examples, the liquid composition comprises a polymer melt or a solution, suspension or emulsion containing the polymer. In some examples, the polymer is a polyurethane. In some examples, the polymer is thermoplastic polyurethane. The term “polyurethane”, as used herein, refers to materials prepared by reaction of a diisocyanate with a polyol. The term refers to a class of polymers composed of organic units joined by carbamate (urethane) links. The term “thermoplastic polyurethane elastomer” or “TPU” refers to an elastomer formed by the inter-reaction of polyols (long-chain dials), di-isocyanates, and short-chain diols. The polyols and the short-chain diols react with the di-isocyanates through polyaddition to form linear polyurethane. The reaction of the polyol with the diisocyanate produces flexible segments of the TPU. The combination of the diisocyanate with the short-chain diols produces rigid segments of the TPU. In some examples, the liquid composition comprises a solution of TPU in dimethylformamide at a TPU concentration in the range of 15% to 25% w/w.

In some examples, formation of a tissue-adherent surface of a leaflet 124 further comprises partially covering a portion of the polymeric surface of the leaflet 124 while exposing another potion thereof, and electrospraying the exposed portion to form an electrosprayed textured layer thereon. In some examples, formation of a tissue-adherent surface of a leaflet 124 further comprises removing the cover after the electrospraying.

Any reference to a leaflet thickness T_{L} throughout the specification and the claims, refers to a thickness of the leaflet 124 excluding electrospun layers, while any electrosprayed layer can have a thickness in addition to the leaflet thickness T_{L}. As shown in FIG. 14A, the leaflet thickness T_{L} is the thickness measured between the leaflet inner surface 160 and the leaflet outer surface 170 prior to forming electrospun layer(s) thereover. Thickness T_{S} denotes the thickness of an electrospun layer that defines a thrombogenic surface. Thickness T_{B} denotes the thickness of an electrospun layer that defines a neointimal-formation encouraging surface. In some examples, thickness T_{S} is greater than thickness T_{B}. In some examples, leaflet thickness T_{L} is between about 10 μm and about 300 μm, including each value and sub-range within this range. In some examples, thickness T_{S} is between about 50 μm and about 200 μm, including each value and sub-range within this range. In some examples, thickness T_{B} is between about 10 μm and about 50 μm, including each value and sub-range within this range.

In some examples, the diameter of electrospun filaments forming a thrombogenic surface is between about 0.1 μm and about 50 μm, including each value and sub-range within this range. In some examples, the diameter of electrospun filaments forming a neointimal-formation encouraging surface is between about 0.1 μm and about 15 μm, including each value and sub-range within this range. In some examples, diameter of electrospun filaments forming a thrombogenic surface is greater than the diameter of electrospun filaments forming a neointimal-formation encouraging surface.

In an electrospun layer, pores having irregular shapes are usually formed between the electrospun filaments. The size of the filaments and the resulting pores can be affected by manufacturing parameters, such as the viscosity of the liquid composition. For example, the liquid viscosity can be in the range of 600 centipoise to 2000 centipoise, 2000 centipoise to 8000 centipoise, or 6000 centipoise to 15000 centipoise. In some examples, the liquid viscosity is in the range of about 7500 centipoise to about 15000 centipoise, including each value and sub-range within this range. Additional manufacturing parameters can include conductivity of the liquid composition, surface tension of the liquid composition, applied voltage during electrospinning, controlling the tip to collector distance (e.g., the distance between the edge of the spinneret and the first face of the leaflet), rate of electrospraying, and/or the temperature of electrospraying.

In some examples, the conductivity of the liquid composition is in the range of 0.5 μS/cm to 1.25 μS/cm. In some examples, the surface tension of the liquid composition is in the range of 30 m/Nm to 50 m/Nm. In some examples, electrospraying of the liquid composition entails applying voltage in the range of 10 kV to 50 kV. In some examples, electrospraying of the liquid composition entails applying voltage in the range of 20 kV to 30 kV. In some examples, electrospraying is performed at a rate of 1 ml/h and 10 ml/h. In some examples, electrospraying is performed at a rate of 3 ml/h and 5 ml/h. In some examples, electrospraying is performed at a temperature of about 15° C. to about 30° C., including each value and sub-range within this range.

Since electrospinning does not enable the pores to be formed in a particular pattern or shape, manufacturing parameters, such as the parameters described above, can be adjusted to ensure that at least a portion of the pores have a certain minimum size and/or a certain maximum size. The size of a pore can be defined as an inscribed circle diameter, which is the diameter of the largest possible circle which can be inscribed in the pore. Stated otherwise, an inscribed circle diameter corresponds to the maximal external diameter of an imaginary cylinder that can be pushed through the pore. In some examples, at least 50% of the pores of a thrombogenic surface have an inscribed circle diameter of about 10 μm to about 100 μm. In some examples, at least 80% of the pores of a thrombogenic surface have an inscribed circle diameter of about 10 μm to about 100 μm. In some examples, at least 50% of the pores of a neointimal-formation encouraging surface have an inscribed circle diameter of about 3 μm to about 50 μm. In some examples, at least 80% of the pores of a neointimal-formation encouraging surface have an inscribed circle diameter of about 3 μm to about 50 μm. In some examples, the averaged inscribed circle diameter of pores of a thrombogenic surface is greater than the averaged inscribed circle diameter of pores of a neointimal-formation encouraging surface.

A polymeric surface termed to be “thromboresistant” throughout the specification and the claims, refers to a surface which is relatively smooth and non-textured, in a manner that will prevent adherence of thromboses thereto, as well as prevent formation of a neointimal tissue layer thereon. A thromboresistant surface can be implemented as a smooth or non-porous surface. In some examples, at least 60% of the surface area of a thromboresistant surface is devoid of pores having an inscribed circle diameter greater than 50 μm. In some examples, at least 60% of the surface area of a thromboresistant surface is devoid of pores having an inscribed circle diameter greater than 10 μm. In some examples, at least 80% of the surface area of a thromboresistant surface is devoid of pores having an inscribed circle diameter greater than 50 μm. In some examples, at least 80% of the surface area of a thromboresistant surface is devoid of pores having an inscribed circle diameter greater than 10 μm.

As shown in FIG. 14D, the leaflet inner surface 160^{h} of exemplary leaflet 124^{h} can also include a proximal inner surface portion 162^{h} implemented as a neointimal-formation encouraging surface, and a distal inner surface portion 164^{h} implemented as a thrombogenic surface. FIG. 15 shows a cross sectional view of an exemplary prosthetic valve 100^{h} comprising a valvular structure 122^{h} that includes leaflets 124^{h} of the type shown in FIG. 14D, representing a potential state of the valve 100^{h} after residing within the implantation site for a certain period of time. The distal outer surface portion 174^{h} and distal inner surface portion 164^{h} can encourage adherence of thromboses 22^{a} and 22^{b}, respectively, thereto, in a manner similar to that described above with respect to FIG. 8B. As further shown in FIG. 15B, a neointimal tissue layer 30a may be formed over the proximal outer surface portion 172^{h}, and a neointimal tissue layer 30b may be formed over the proximal inner surface portion 162^{h}.

While illustrated in FIG. 14D to include a combination of a proximal outer surface portion 172^{h} and a proximal inner surface portion 162^{h}, implemented as neointimal-formation encouraging surfaces, as well as a distal outer surface portion 174^{h} and a distal inner surface portion 164^{h}, implemented as thrombogenic surfaces, it is to be understood that this is shown by way of illustration and not limitation, and that any other combination of surfaces and/or surface portions is contemplated. In some examples, a leaflet 124 can have a leaflet outer surface 170 that includes a proximal outer surface portion 172 implemented as a neointimal-formation encouraging surface, and a distal outer surface portion 174 implemented as a thrombogenic surface, while the leaflet inner surface 160 can be relatively smooth (without including any tissue-adherent surfaces), or can include a distal inner surface portion 164 implemented as a thrombogenic surface and a proximal inner surface portion 162 which is thromboresistant.

In some examples, a leaflet 124 can have a leaflet inner surface 160 that includes a proximal inner surface portion 162 implemented as a neointimal-formation encouraging surface, and a distal inner surface portion 164 implemented as a thrombogenic surface, while the leaflet outer surface 170 can be relatively smooth (without including any tissue-adherent surfaces), or can include a distal outer surface portion 174 implemented as a thrombogenic surface and a proximal outer surface portion 172 which is thromboresistant.

It is to be understood that formation of polymeric porous tissue-adherent surfaces is not limited to electrospinning, but may be rather achieved by any other suitable manufacturing procedure that forms pores having inscribed circle diameters in the ranges mentioned above for thrombogenic and/or neointimal-formation encouraging surfaces.

In some examples, a tissue-adherent surface of a leaflet 124 is formed by salt leaching. In general, salt leaching is a method that produces porous media, including porous polymeric materials. When used to form porous polymers, salt leaching procedures involve production of a liquid mixture of a polymer melt or solution in its pre-casted form. The mixture further comprises a porogen, which is the compound that will eventually form the pores. As an organic material, the pre-casted polymer is soluble in organic solvents and insoluble in water. The porogen is selected such that it has the opposite solubility properties, e.g., it is a solid which is insoluble in organic solvents (and/or in the polymer melt) and is soluble in water. The mixture of the liquid (melted or solubilized) polymer and the porogen includes, therefore, liquid with solid particles of the porogen therein. In some examples, the liquid mixture is made substantially homogeneous upon mixing, e.g., the solid particles a scattered substantially evenly in the liquid. The mixture is then casted or molded and hardened to form the desired shape of the product polymer. In this stage, the composition is of a solid polymer mixed with particles of the porogen. The composition is then thoroughly washed with water to dissolve and leach the porogen, and leave the polymer with the remaining vacant pores.

The liquid composition of the polymer can be similar to any example described above for a liquid composition used in an electrospinning procedure. In some examples, formation of a tissue-adherent surface of a leaflet 124 comprises providing a porogen, which can be solid at room temperature. In some examples, the porogen has melting point above 100° C. or above 200° C. In some examples, the porogen is an inorganic compound. In some examples, the porogen has aqueous solubility of at least 1 g/mL, at least 5 g/mL or at least 10 g/mL. In some examples, the porogen is in the form of a powder. In some examples, the porogen has particle size in the range of 0.4 μm to 4 μm, 2 μm to 20 μm, 10 μm to 100 μm, 50 μm to 500 μm, 100 μm to 1000 μm, or 200 μm to 2000 μm. In some examples, formation of a tissue-adherent surface of a leaflet 124 comprises forming a textured surface along a polymeric surface of the leaflet 124 by salt leaching using powder (SLUP).

In some examples, the porogen is selected from the group consisting of: sodium chloride, potassium chloride, sodium bicarbonate, and ammonium bicarbonate. In some examples, the porogen comprises sodium chloride.

In some examples, formation of a tissue-adherent surface of a leaflet 124 comprises mixing the liquid composition of the polymer with the porogen. The formed mixture can comprise the solid porogen undissolved in the liquid composition. In some examples, the mixture can be substantially homogenous.

In some examples, formation of a tissue-adherent surface of a leaflet 124 by salt leaching comprises casting the mixture to form a solid casted layer that includes the porogen dispersed within the casted solid polymer. In some examples, formation of a tissue-adherent surface of a leaflet 124 by salt leaching comprises molding the mixture to form a solid molded layer that comprises the porogen dispersed within the molded solid polymer. In some examples, the porogen particles can be substantially evenly dispersed within the solid polymer.

In some examples, formation of a tissue-adherent surface of a leaflet 124 by salt leaching further comprises contacting the formed solid layer with water, thereby leaching at least some of the porogen from the solid layer to form vacated pores within the layer. In some examples, contact with water can be performed at a temperature of at least 20° C., at least 25° C., at least 30° C., at least 40° C., at least 60° C., or at least 80° C. In some examples, contact with water can be performed for a period of 1 to 120 minutes.

In some examples, a solid textured layer can be separately formed by salt leaching, and then attached to a polymeric surface of leaflet 124. Such an attachment can be achieved by any suitable manner, such as gluing, fusing, and the like. For example, formation of the leaflet can include providing the polymer from which the leaflet is to be formed as a melt, and casting the polymer, wherein during hardening of the polymer, a solid leached layer is fused to the hardening leaflet. It is to be understood that in this context, “solid” includes polymers which are flexible, as long as they are not liquid.

In some examples, a solid textured layer formed by salt leaching is not formed as a separate layer that needs to be attached to a surface of the leaflet, but is formed directly over the polymeric leaflet, or a portion thereof along which a tissue-adherent surface is to be formed.

In some examples, such as when salt leaching is used to form a tissue-adherent surface as an integral surface of the leaflet as a whole, or when a salt-leached surface is fused to a surface of a leaflet, no additional layer thickness is added, such that the leaflet thickness T_{L} remains substantially the same along the tissue-adherent surface(s) as well, while the pores may extend into the leaflet thickness T_{L}, but without extending through the whole thickness of the leaflet T_{L}. Thus, while thrombogenic surfaces such as surfaces 174^{h} and 164^{h} are illustrated in FIGS. 14A-14D to have an additional layer thickness T_{S}, and neointimal-formation encouraging surfaces such as surfaces 172^{h} and 162^{h} are illustrated in FIGS. 14A-14D to have an additional layer thickness T_{B}, it is to be understood that this is shown by way of illustration and not limitation, and that in some examples, such surfaces will not add thickness to the leaflet, but may rather include pores extending into the polymeric material forming leaflet thickness T_{L}. The pores along tissue-adherent surfaces formed by salt leaching can have the same inscribed circle diameter as described in any example above.

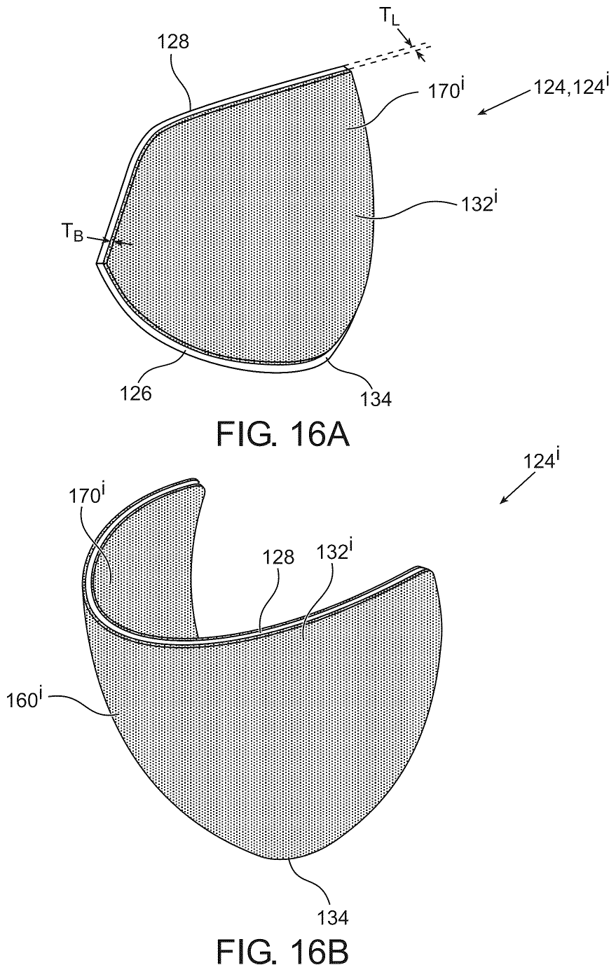

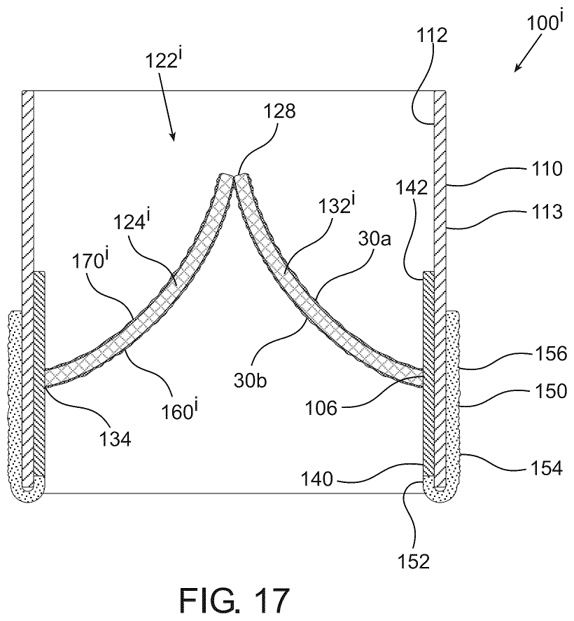

In some examples, a polymeric surface of a leaflet 124, such as leaflet inner surface 160 and/or leaflet outer surface 170, is implemented as a neointimal-formation encouraging surface, without necessarily including a differently-textured portion, such as a thrombogenic surface. FIGS. 16A-16B show views in perspective from different angles of view, of an exemplary leaflet 124ⁱ. Leaflet 124ⁱ is an exemplary implementation of leaflet 124, and thus includes all of the features described for leaflet 124 throughout the current disclosure, except that at least one surface of leaflet 124 is implemented as a neointimal-formation encouraging surface, without including a thrombogenic surface portion. In the illustrated example, both the leaflet outer surface 170^{h} and the leaflet inner surface 160^{h} are shown to be uniformly textured, implemented as neointimal-formation encouraging surfaces. FIG. 17 shows a cross sectional view of an exemplary prosthetic valve 100ⁱ comprising a valvular structure 122ⁱ that includes leaflets 124ⁱ of the type shown in FIGS. 16A-16B, representing a potential state of the valve 100ⁱ after residing within the implantation site for a certain period of time.

Each of the neointimal-formation encouraging surfaces 160ⁱ and 170ⁱ can have a thickness T_{B} as illustrated, for example when formed by electrospinning procedures. However, it is to be understood that the thickness T_{B} is shown in FIGS. 16A-16B by way of illustration and not limitation, and that in some examples, the leaflet 124ⁱ can have a uniform thickness T_{L}, for example when formed by salt leaching procedures. As shown in FIG. 17, a neointimal tissue layer 30^{a} may be formed over the entire area of the leaflet outer surface 170ⁱ, and a neointimal tissue layer 30b may be formed over the entire area of the leaflet inner surface 160ⁱ.

While illustrated in FIG. 16B to include both leaflet outer surface 170 and a leaflet inner surface 160 implemented as uniform neointimal-formation encouraging surfaces that cover the entire area of these surfaces, it is to be understood that this is shown by way of illustration and not limitation, and that any other combination of surfaces and/or surface portions is contemplated. In some examples, a leaflet 124 can have a leaflet outer surface 170 implemented as a neointimal-formation encouraging surface devoid of a thrombogenic surface, while the leaflet inner surface 160 can be entirely thromboresistant, or can include a distal inner surface portion 164 implemented as a thrombogenic surface, and a proximal inner surface portion 162 that can be either thromboresistant or implemented as a neointimal-formation encouraging surface.

In some examples, a leaflet 124 can have a leaflet inner surface 160 implemented as a neointimal-formation encouraging surface devoid of a thrombogenic surface, while the leaflet outer surface 170 can be entirely thromboresistant, or can include a distal outer surface portion 174 implemented as a thrombogenic surface, and a proximal outer surface portion 172 that can be either thromboresistant or implemented as a neointimal-formation encouraging surface.

While exemplary distal outer surface portions 174 and/or distal inner surface portions 164, when implemented as thrombogenic surfaces, are illustrated throughout FIGS. 5A, 7 and 9, without an added layer thickness T_{S}, it is to be understood that any of such thrombogenic surfaces can have a layer thickness T_{S}, such as illustrated in FIGS. 14A-14B, for example when formed by electrospinning procedures, or can have pores extending into the thickness T_{L} of the leaflet, without adding additional layer thickness, for example when formed by selected exemplary salt leaching procedures.

In some examples, when a leaflet includes both a distal outer surface portions 174 and a distal inner surface portions 164, as illustrated in FIG. 7, 10 or 14B for example, the pores of each of these surfaces can be differently sized, while still meeting exemplary ranges described above for pore sizes of thrombogenic surfaces. In some examples, the averaged inscribed circle diameter of the pores of a distal outer surface portion 174 is greater than the averaged inscribed circle diameter of the pores of a distal inner surface portion 164. Similarly, when such thrombogenic surfaces are formed to have a layer thickness T_{S}, the thicknesses T_{S} can be equal or different. In some examples, the layer thickness T_{S} of the distal outer surface portion 174 is greater than that of the distal inner surface portion 164.

In some examples, when both the leaflet outer surface 170 and the leaflet inner surface 160 include neointimal-formation encouraging surfaces, as illustrated in FIG. 14B or 16B for example, the pores of each of these surfaces can be differently sized, while still meeting exemplary ranges described above for pore sizes of neointimal-formation encouraging surfaces. In some examples, the averaged inscribed circle diameter of the pores of a neointimal-formation encouraging surface formed along a proximal outer surface portion 172 or the entire surface area of leaflet outer surface 170, is greater than the averaged inscribed circle diameter of the pores of a neointimal-formation encouraging surface formed along a proximal inner surface portion 162 or the entire surface area of leaflet inner surface 160. Similarly, when such thrombogenic surfaces are formed to have a layer thickness T_{S}, the thicknesses T_{S} can be equal or different. In some examples, the layer thickness T_{S} of a neointimal-formation encouraging surface formed along a proximal outer surface portion 172 or the entire surface area of leaflet outer surface 170, is greater than that of a neointimal-formation encouraging surface formed along a proximal inner surface portion 162 or the entire surface area of leaflet inner surface 160.

While various examples of prosthetic valve 100 are described above and illustrated throughout some of the drawings to include an outer skirt 150 configured to allow tissue ingrowth, it is to be understood that in some examples, the outer skirt 150 does not include features that encourage such ingrowth.

While various examples of prosthetic valve 100 are described above and illustrated throughout some of the drawings to include an outer skirt 150, it is to be understood that in some examples, a prosthetic valve 100 can be devoid of an outer skirt.

### Some Examples of the Disclosed Implementations

Some examples of above-described implementations are enumerated below. It should be noted that one feature of an example in isolation or more than one feature of the example taken in combination and, optionally, in combination with one or more features of one or more examples below are examples also falling within the disclosure of this application.

Example 1. A prosthetic valve comprising: a frame movable between a radially compressed state and a radially expanded state; a valvular structure mounted inside the frame and comprising a plurality of leaflets, wherein each leaflet comprises a cusp edge, a free edge opposite to the cusp edge, and leaflet polymeric surfaces extending between the cusp edge and the free edge, the leaflet polymeric surfaces comprising: a leaflet polymeric inner surface, facing a central longitudinal axis of the prosthetic valve; and a leaflet polymeric outer surface, facing the frame; wherein at least one of the leaflet polymeric surfaces comprises at least one tissue-adherent surface configured to allow tissue adherence thereto.

Example 2. The prosthetic valve of any example herein, particularly of example 1, wherein the leaflet polymeric surfaces comprise thermoplastic polyurethane.

Example 3. The prosthetic valve of any example herein, particularly of example 1 or 2, wherein the at least one tissue-adherent surface comprises thermoplastic polyurethane.

Example 4. The prosthetic valve of any one of any example herein, particularly of examples 1 to 3, wherein the at least one tissue-adherent surface comprises a plurality of pores.

Example 5. The prosthetic valve of any example herein, particularly of example 4, wherein the at least one tissue-adherent surface comprises at least one thrombogenic surface configured to allow adherence of tissue exceeding an average thickness of 200 μm thereto.

Example 6. The prosthetic valve of any example herein, particularly of example 5, wherein the thrombogenic surface is configured to prevent spontaneous detachment of tissue adhered thereto for at least a predetermined period of time.

Example 7. The prosthetic valve of any example herein, particularly of example 6, wherein the predetermined period of time is at least one month.

Example 8. The prosthetic valve of any example herein, particularly of example 6, wherein the predetermined period of time is at least six months.

Example 9. The prosthetic valve of any example herein, particularly of example 6, wherein the predetermined period of time is at least one year.

Example 10. The prosthetic valve of any one of any example herein, particularly of examples 5 to 9, wherein at least 50% of the plurality of pores of the thrombogenic surface have an inscribed circle diameter of between 10 μm and 100 μm.

Example 11. The prosthetic valve of any one of any example herein, particularly of examples 5 to 9, wherein at least 80% of the plurality of pores of the thrombogenic surface have an inscribed circle diameter of between 10 μm and 100 μm.

Example 12. The prosthetic valve of any one of any example herein, particularly of examples 5 to 11, wherein the pores of the thrombogenic surface are formed by a process of salt leaching.

Example 13. The prosthetic valve of any one of any example herein, particularly of examples 5 to 11, wherein the pores of the thrombogenic surface are formed between electrospun filaments.

Example 14. The prosthetic valve of any example herein, particularly of example 13, wherein the electrospun filaments forming the thrombogenic surface have diameters between 0.1 μm and 50 μm.

Example 15. The prosthetic valve of any example herein, particularly of example 13 or 14, wherein an electrospun layer defining the thrombogenic surface has a thickness between 50 μm and 200 μm.

Example 16. The prosthetic valve of any one of any example herein, particularly of examples 5 to 15, wherein the leaflet polymeric outer surface comprises a distal outer surface portion extending between the cusp edge and a proximal edge of the distal outer surface portion, and a proximal outer surface portion extending between the proximal edge of the distal outer surface portion and the free edge.

Example 17. The prosthetic valve of any example herein, particularly of example 16, wherein the distal outer surface portion comprises the at least one thrombogenic surface.

Example 18. The prosthetic valve of any example herein, particularly of example 16 or 17, wherein the distal outer surface portion defines a textured outer portion height, which is less than a height defined by the proximal outer surface portion.

Example 19. The prosthetic valve of any example herein, particularly of example 18, wherein the height defined by the proximal outer surface portion is at least two times greater than the textured outer portion height.

Example 20. The prosthetic valve of any example herein, particularly of example 18, wherein the height defined by the proximal outer surface portion is at least three times greater than the textured outer portion height.

Example 21. The prosthetic valve of any example herein, particularly of example 18, wherein the height defined by the proximal outer surface portion is at least four times greater than the textured outer portion height.

Example 22. The prosthetic valve of any one of any example herein, particularly of examples 18 to 21, wherein the textured outer portion height has a maximum value at a position of a cusp edge midpoint of the cusp.

Example 23. The prosthetic valve of any example herein, particularly of example 22, wherein the value of the textured outer portion height at proximal ends of the distal outer surface portion, is less than the maximum value of the textured outer portion height at the position of the cusp edge midpoint.

Example 24. The prosthetic valve of any example herein, particularly of example 22 or 23, wherein the maximum value of the textured outer portion height is equal to or less than 20% of a radius of the prosthetic valve in the radially expanded state.

Example 25. The prosthetic valve of any example herein, particularly of example 24, wherein the maximum value of the textured outer portion height is equal to or less than 10% of the radius of the prosthetic valve in the radially expanded state.

Example 26. The prosthetic valve of any one of any example herein, particularly of examples 22 to 25, wherein the maximum value of the textured outer portion height is between 1 mm and 3 mm.

Example 27. The prosthetic valve of any one of any example herein, particularly of examples 16 to 21, wherein the proximal edge of the distal outer surface portion is parallel to the cusp edge.

Example 28. The prosthetic valve of any one of any example herein, particularly of examples 16 to 27, wherein the proximal outer surface portion is thromboresistant.

Example 29. The prosthetic valve of any example herein, particularly of example 28, wherein at least 60% of the surface area of the proximal outer surface portion is devoid of pores having an inscribed circle diameter greater than 50 μm.

Example 30. The prosthetic valve of any example herein, particularly of example 28, wherein at least 60% of the surface area of the proximal outer surface portion is devoid of pores having an inscribed circle diameter greater than 10 μm.

Example 31. The prosthetic valve of any example herein, particularly of example 28, wherein at least 80% of the surface area of a thromboresistant surface is devoid of pores having an inscribed circle diameter greater than 50 μm.

Example 32. The prosthetic valve of any example herein, particularly of example 28, wherein at least 60% of the surface area of the proximal outer surface portion is devoid of pores having an inscribed circle diameter greater than 10 μm.

Example 33. The prosthetic valve of any one of any example herein, particularly of examples 16 to 27, wherein the at least one tissue-adherent surface comprises at least one neointimal-formation encouraging surface configured to allow formation of neointimal tissue that does not exceed an average thickness of 200 μm.

Example 34. The prosthetic valve of any example herein, particularly of example 33, wherein the proximal outer surface portion comprises the at least one neointimal-formation encouraging surface.

Example 35. The prosthetic valve of any example herein, particularly of example 34, wherein at least 50% of the plurality of pores of the proximal outer surface portion have an inscribed circle diameter of between 3 μm and 50 μm.

Example 36. The prosthetic valve of any example herein, particularly of example 34, wherein at least 80% of the plurality of pores of the proximal outer surface portion have an inscribed circle diameter of between 3 μm and 50 μm.

Example 37. The prosthetic valve of any one of any example herein, particularly of examples 34 to 36, wherein the pores of the proximal outer surface portion are formed by a process of salt leaching.

Example 38. The prosthetic valve of any one of any example herein, particularly of examples 34 to 36, wherein the pores of the proximal outer surface portion are formed between electrospun filaments.

Example 39. The prosthetic valve of any example herein, particularly of example 38, wherein the electrospun filaments forming the proximal outer surface portion have diameters between 0.1 μm and 15 μm.

Example 40. The prosthetic valve of any example herein, particularly of example 38 or 39, wherein an electrospun layer defining the proximal outer surface portion has a thickness between 10 μm and 50 μm.

Example 41. The prosthetic valve of any one of any example herein, particularly of examples 35 to 40, wherein an averaged inscribed circle diameter of the pores of the distal outer surface portion is greater than an averaged inscribed circle diameter of the pores of the proximal outer surface portion.

Example 42. The prosthetic valve of any one of any example herein, particularly of examples 5 to 32, wherein the leaflet polymeric inner surface comprises a distal inner surface portion extending between the cusp edge and a proximal edge of the distal inner surface portion, and a proximal inner surface portion extending between the proximal edge of the distal inner surface portion and the free edge.

Example 43. The prosthetic valve of any example herein, particularly of example 42, wherein the distal inner surface portion comprises the at least one thrombogenic surface.

Example 44. The prosthetic valve of any example herein, particularly of example 42 or 43, wherein the proximal edge of the distal inner surface portion is parallel to the cusp edge.

Example 45. The prosthetic valve of any example herein, particularly of example 42 or 43, wherein the distal inner surface portion defines a textured inner portion height, having a value at proximal ends of the distal inner surface portions that is less than the value of the textured inner portion height at the position of a cusp edge midpoint of the cusp edge.

Example 46. The prosthetic valve of any one of any example herein, particularly of examples 42 to 45, wherein the proximal inner surface portion is thromboresistant.

Example 47. The prosthetic valve of any example herein, particularly of example 46, wherein at least 60% of the surface area of the proximal inner surface portion is devoid of pores having an inscribed circle diameter greater than 50 μm.

Example 48. The prosthetic valve of any example herein, particularly of example 46, wherein at least 60% of the surface area of the proximal inner surface portion is devoid of pores having an inscribed circle diameter greater than 10 μm.

Example 49. The prosthetic valve of any example herein, particularly of example 46, wherein at least 60% of the surface area of the proximal inner surface portion is devoid of pores having an inscribed circle diameter greater than 50 μm.

Example 50. The prosthetic valve of any example herein, particularly of example 46, wherein at least 60% of the surface area of the proximal inner surface portion is devoid of pores having an inscribed circle diameter greater than 10 μm.

Example 51. The prosthetic valve of any one of any example herein, particularly of examples 42 to 50, wherein the at least one tissue-adherent surface comprises at least one neointimal-formation encouraging surface configured to allow formation of neointimal tissue that does not exceed an average thickness of 200 μm.

Example 52. The prosthetic valve of any example herein, particularly of example 51, wherein the proximal inner surface portion comprises the at least one neointimal-formation encouraging surface.

Example 53. The prosthetic valve of any example herein, particularly of example 52, wherein at least 50% of the plurality of pores of the proximal inner surface portion have an inscribed circle diameter of between 3 μm and 50 μm.

Example 54. The prosthetic valve of any example herein, particularly of example 52, wherein at least 80% of the plurality of pores of the proximal inner surface portion have an inscribed circle diameter of between 3 μm and 50 μm.

Example 55. The prosthetic valve of any one of any example herein, particularly of examples 52 to 54, wherein the pores of the proximal inner surface portion are formed by a process of salt leaching.

Example 56. The prosthetic valve of any one of any example herein, particularly of examples 52 to 55, wherein the pores of the proximal inner surface portion are formed between electrospun filaments.

Example 57. The prosthetic valve of any example herein, particularly of example 56, wherein the electrospun filaments forming the proximal inner surface portion have diameters between 0.1 μm and 15 μm.

Example 58. The prosthetic valve of any example herein, particularly of example 56 or 57, wherein an electrospun layer defining the proximal inner surface portion has a thickness between 10 μm and 50 μm.

Example 59. The prosthetic valve of any one of any example herein, particularly of examples 53 to 58, wherein an averaged inscribed circle diameter of the pores of the distal inner surface portion is greater than an averaged inscribed circle diameter of the pores of the proximal inner surface portion.

Example 60. The prosthetic valve of any one of any example herein, particularly of examples 18 to 26, wherein the leaflet polymeric inner surface comprises a distal inner surface portion defining a textured inner portion height between the cusp edge and a proximal edge of the distal inner surface portion, and a proximal inner surface portion extending between the proximal edge of the distal inner surface portion and the free edge.

Example 61. The prosthetic valve of any example herein, particularly of example 60, wherein the distal inner surface portion comprises the at least one thrombogenic surface.

Example 62. The prosthetic valve of any example herein, particularly of example 60 or 61, wherein the textured inner portion height is equal to the textured outer portion height.

Example 63. The prosthetic valve of any example herein, particularly of example 60 or 61, wherein the textured outer portion height is greater than the textured inner portion height.

Example 64. The prosthetic valve of any example herein, particularly of example 63, wherein the textured outer portion height is at least twice as great as the textured inner portion height.

Example 65. The prosthetic valve of any example herein, particularly of example 4, wherein the at least one tissue-adherent surface comprises at least one neointimal-formation encouraging surface configured to allow formation of neointimal tissue that does not exceed an average thickness of 200 μm.

Example 66. The prosthetic valve of any example herein, particularly of example 65, wherein the leaflet polymeric outer surface comprises the at least one neointimal-formation encouraging surface.

Example 67. The prosthetic valve of any example herein, particularly of example 66, wherein the entire surface area of the leaflet polymeric outer surface comprises the at least one neointimal-formation encouraging surface.

Example 68. The prosthetic valve of any one of any example herein, particularly of examples 65 to 67, wherein the leaflet polymeric inner surface comprises the at least one neointimal-formation encouraging surface.

Example 69. The prosthetic valve of any example herein, particularly of example 68, wherein the entire surface area of the leaflet polymeric inner surface comprises the at least one neointimal-formation encouraging surface.

Example 70. The prosthetic valve of any one of any example herein, particularly of examples 65 to 69, wherein at least 50% of the plurality of pores of the at least one neointimal-formation encouraging surface have an inscribed circle diameter of between 3 μm and 50 μm.

Example 71. The prosthetic valve of any one of any example herein, particularly of examples 65 to 69, wherein at least 80% of the plurality of pores of the at least one neointimal-formation encouraging surface have an inscribed circle diameter of between 3 μm and 50 μm.

Example 72. The prosthetic valve of any one of any example herein, particularly of examples 65 to 71, wherein the pores of the at least one neointimal-formation encouraging surface are formed by a process of salt leaching.

Example 73. The prosthetic valve of any one of any example herein, particularly of examples 65 to 71, wherein the pores of the at least one neointimal-formation encouraging surface are formed between electrospun filaments.

Example 74. The prosthetic valve of any example herein, particularly of example 73, wherein the electrospun filaments forming the at least one neointimal-formation encouraging surface have diameters between 0.1 μm and 15 μm.

Example 75. The prosthetic valve of any example herein, particularly of example 73 or 74, wherein an electrospun layer defining the at least one neointimal-formation encouraging surface has a thickness between 10 μm and 50 μm.

Example 76. The prosthetic valve of any one of any example herein, particularly of examples 1 to 75, wherein each leaflet has a leaflet thickness between 10 μm and 300 μm.

Example 77. The prosthetic valve of any one of any example herein, particularly of examples 1 to 75, wherein each leaflet comprises a base layer between the leaflet polymeric inner surface and the leaflet polymeric outer surface.

Example 78. The prosthetic valve of any example herein, particularly of example 77, wherein the base layer comprises a polymeric material which is different from the polymeric material comprised in the leaflet polymeric surfaces.

Example 79. The prosthetic valve of any example herein, particularly of example 77, wherein the base layer comprises a fabric.

Example 80. The prosthetic valve of any example herein, particularly of example 77, wherein the base layer comprises a tissue.

Example 81. The prosthetic valve of any one of any example herein, particularly of examples 1 to 80, wherein the valvular structure is coupled to the frame along a scalloped line.

Example 82. The prosthetic valve of any one of any example herein, particularly of examples 1 to 80, further comprising an inner skirt coupled to an inner surface of the frame, the inner skirt defining a skirt internal surface facing the central longitudinal axis.

Example 83. The prosthetic valve of any example herein, particularly of example 82, wherein the skirt internal surface is thromboresistant.

Example 84. The prosthetic valve of any example herein, particularly of example 82 or 83, wherein the valvular structure is coupled to the skirt internal surface along a scalloped line.

Example 85. The prosthetic valve of any example herein, particularly of example 82 or 83, further comprising a strip attached to skirt internal surface and defining a strip internal surface facing the central longitudinal axis.

Example 86. The prosthetic valve of any one of any example herein, particularly of examples 1 to 80, further comprising a strip coupled to an inner surface of the frame, the strip defining a strip internal surface facing the central longitudinal axis.

Example 87. The prosthetic valve of any example herein, particularly of example 85 or 86, wherein the strip is configured to allow tissue adherence to the strip internal surface.

Example 88. The prosthetic valve of any one of any example herein, particularly of examples 85 to 87, wherein the valvular structure is coupled to the strip internal surface along a scalloped line.

Example 89. The prosthetic valve of any example herein, particularly of example 88, wherein the strip comprises a proximal portion extending between the scalloped line and a proximal edge of the strip.

Example 90. The prosthetic valve of any example herein, particularly of example 89, wherein the strip further comprises a distal portion extending between the scalloped line and a distal edge of the strip.

Example 91. The prosthetic valve of any one of any example herein, particularly of examples 85 to 90, wherein the strip comprises yarns extending radially inward.

Example 92. The prosthetic valve of any one of any example herein, particularly of examples 85 to 90, wherein the strip comprises polyethylene terephthalate.

Example 93. The prosthetic valve of any one of any example herein, particularly of examples 85 to 90, wherein the strip comprises a knit pattern formed along the strip internal surface.

Example 94. The prosthetic valve of any one of any example herein, particularly of examples 85 to 90, wherein the strip comprises a porous texture.

Example 95. The prosthetic valve of any one of any example herein, particularly of examples 85 to 94, wherein the strip is configured to allow adherence of tissue exceeding an average thickness of 200 μm to the strip internal surface.

Example 96. The prosthetic valve of any one of any example herein, particularly of examples 1 to 95, wherein the plurality of leaflet comprises three leaflets.

Example 97. The prosthetic valve of any one of any example herein, particularly of examples 1 to 96, further comprising an outer skirt coupled to an outer surface of the frame.

Example 98. The prosthetic valve of any example herein, particularly of example 97, wherein the outer skirt is configured to allow tissue ingrowth.

Example 99. The prosthetic valve of any example herein, particularly of example 98, wherein the outer skirt comprises yarns extending radially outward.

Example 100. The prosthetic valve of any example herein, particularly of example 98, wherein the outer skirt comprises polyethylene terephthalate.

Example 101. The prosthetic valve of any example herein, particularly of example 98, wherein the outer skirt comprises a knit pattern formed along an external surface thereof.

Example 102. The prosthetic valve of any example herein, particularly of example 98, wherein the outer skirt comprises a porous texture.

Example 103. The prosthetic valve of any one of any example herein, particularly of examples 97 to 102, wherein the outer skirt is a bioresorbable outer skirt.

Example 104. A prosthetic valve comprising: a frame movable between a radially compressed state and a radially expanded state; and a leaflet coupled to the frame and comprising a first edge, a second edge opposite to the first edge, and polymeric surfaces extending between the first and second edges, wherein at least one of the leaflet polymeric surfaces comprises at least one tissue-adherent surface, configured to allow tissue adherence thereto.

Example 105. A leaflet for a prosthetic heart valve, the leaflet comprising: a cusp edge; a free edge opposite to the cusp edge; and leaflet polymeric surfaces extending between the cusp edge and the free edge, the leaflet polymeric surfaces comprising at least one tissue-adherent surface configured to allow tissue adherence thereto.

It is appreciated that certain features of the disclosure, which are, for clarity, described in the context of separate examples, may also be provided in combination in a single example. Conversely, various features of the disclosure, which are, for brevity, described in the context of a single example, may also be provided separately or in any suitable sub-combination or as suitable in any other described example of the disclosure. No feature described in the context of an example is to be considered an essential feature of that example, unless explicitly specified as such.

In view of the many possible examples to which the principles of the disclosure may be applied, it should be recognized that the illustrated examples are only preferred examples and should not be taken as limiting the scope. Rather, the scope is defined by the following claims. We therefore claim all that comes within the scope and spirit of these claims.

What is claimed is:

1. A prosthetic valve comprising:
a frame movable between a radially compressed state and a radially expanded state;
a valvular structure mounted inside the frame and comprising a plurality of leaflets, wherein each leaflet comprises a cusp edge, a free edge opposite to the cusp edge, and leaflet polymeric surfaces extending between the cusp edge and the free edge, the leaflet polymeric surfaces comprising:
a leaflet polymeric inner surface, facing a central longitudinal axis of the prosthetic valve; and
a leaflet polymeric outer surface, facing the frame;
wherein at least one of the leaflet polymeric surfaces comprises at least one tissue-adherent surface configured to allow tissue adherence thereto.

2. The prosthetic valve of claim 1, wherein the at least one tissue-adherent surface comprises thermoplastic polyurethane.

3. The prosthetic valve of claim 1, wherein the at least one tissue-adherent surface comprises a plurality of pores.

4. The prosthetic valve of claim 3, wherein the at least one tissue-adherent surface comprises at least one thrombogenic surface configured to allow adherence of tissue exceeding an average thickness of 200 μm thereto.

5. The prosthetic valve of claim 4, wherein the thrombogenic surface is configured to prevent spontaneous detachment of tissue adhered thereto for at least a predetermined period of time.

6. The prosthetic valve of claim 4, wherein at least 80% of the plurality of pores of the thrombogenic surface have an inscribed circle diameter of between 10 μm and 100 μm.

7. The prosthetic valve of claim 4, wherein the pores of the thrombogenic surface are formed between electrospun filaments.

8. The prosthetic valve of claim 7, wherein an electrospun layer defining the thrombogenic surface has a thickness between 50 μm and 200 μm.

9. The prosthetic valve of claim 4, wherein the leaflet polymeric outer surface comprises a distal outer surface portion extending between the cusp edge and a proximal edge of the distal outer surface portion, and a proximal outer surface portion extending between the proximal edge of the distal outer surface portion and the free edge.

10. The prosthetic valve of claim 9, wherein the distal outer surface portion defines a textured outer portion height, which is less than a height defined by the proximal outer surface portion.

11. The prosthetic valve of claim 9, wherein the at least one tissue-adherent surface comprises at least one neointimal-formation encouraging surface configured to allow formation of neointimal tissue that does not exceed an average thickness of 200 μm.

12. The prosthetic valve of claim 3, wherein the at least one tissue-adherent surface comprises at least one neointimal-formation encouraging surface configured to allow formation of neointimal tissue that does not exceed an average thickness of 200 μm.

13. The prosthetic valve of claim 12, wherein the leaflet polymeric outer surface comprises the at least one neointimal-formation encouraging surface.

14. The prosthetic valve of claim 13, wherein the entire surface area of the leaflet polymeric outer surface comprises the at least one neointimal-formation encouraging surface.

15. The prosthetic valve of claim 12, wherein the leaflet polymeric inner surface comprises the at least one neointimal-formation encouraging surface.

16. The prosthetic valve of claim 15, wherein the entire surface area of the leaflet polymeric inner surface comprises the at least one neointimal-formation encouraging surface.

17. The prosthetic valve of claim 12, wherein at least 80% of the plurality of pores of the at least one neointimal-formation encouraging surface have an inscribed circle diameter of between 3 μm and 50 μm.

18. The prosthetic valve of claim 12, wherein the pores of the at least one neointimal-formation encouraging surface are formed between electrospun filaments.

19. The prosthetic valve of claim 18, wherein an electrospun layer defining the at least one neointimal-formation encouraging surface has a thickness between 10 μm and 50 μm.

20. The prosthetic valve of claim 1, wherein each leaflet comprises a base layer between the leaflet polymeric inner surface and the leaflet polymeric outer surface.

21. The prosthetic valve of claim 20, wherein the base layer comprises a tissue.
